(19) United States
(12) Patent Application Publication
(10) Pub. No.: US 2025/0144110 A1
(43) Pub. Date: May 8, 2025

(54) COMPOSITIONS OF AUTOPHAGY MODULATING AGENTS AND USES THEREOF

(71) Applicant: Washington University, St. Louis, MO (US)

(72) Inventors: David Perlmutter, St. Louis, MO (US); Roland Dolle, St. Louis, MO (US); Stephen Pak, St. Louis, MO (US); Gary Silverman, St. Louis, MO (US)

(73) Assignee: Washington University, St. Louis, MO (US)

(21) Appl. No.: 18/833,591

(22) PCT Filed: Jan. 27, 2023
(86) PCT No.: PCT/US2023/061507

§ 371 Date: Jul. 26, 2024

Related U.S. Application Data
(60) Provisional application No. 63/304,151, filed on Jan. 28, 2022.

(51) Int. Cl. A61K 31/5415; A61K 31/4375; A61K 31/4704; A61K 31/4709; A61K 31/473; A61K 31/4741; A61K 31/4745; A61K 31/496; A61K 31/498; A61K 31/5025; A61K 31/506; A61K 31/517; A61K 31/538

(52) U.S. Cl. CPC A61K 31/5415; A61K 31/4375; A61K 31/4704; A61K 31/4709; A61K 31/473; A61K 31/4741; A61K 31/4745; A61K 31/496; A61K 31/498; A61K 31/5025; A61K 31/506; A61K 31/517; A61K 31/538

(57) ABSTRACT

Among the various aspects of the present disclosure is the provision of compositions and methods for modulating autophagy for treatment and prevention of autophagy-associated diseases, disorders, or conditions and compositions of photoreactive compounds. An aspect of the present disclosure provides for autophagy enhancing agents capable of treating or preventing diseases, disorders, or conditions in which autophagy is implicated.

### CROSS-REFERENCE TO RELATED APPLICATIONS

This application claims priority from U.S. Provisional Application Ser. No. 63/304,151 filed on 28 Jan. 2022, which is incorporated herein by reference in its entirety.

### STATEMENT REGARDING FEDERALLY SPONSORED RESEARCH OR DEVELOPMENT

This invention was made with government support under DK096990 and DK104946 awarded by the National Institutes of Health. The government has certain rights in the invention.

### MATERIAL INCORPORATED-BY-REFERENCE

Not applicable.

### FIELD

The present disclosure generally relates to methods of modulating autophagy in subjects suffering from autophagy-associated diseases, disorders, or conditions.

### SUMMARY

Among the various aspects of the present disclosure is the provision of compositions and methods of modulating autophagy for the treatment of autophagy-associated diseases and compositions of photoreactive compounds.

An aspect of the present disclosure provides for compositions and methods for modulating autophagy or treating or preventing an autophagy-associated disease, disorder, or condition in a subject in need thereof. In some embodiments, the method comprises administering to the subject a therapeutically effective amount of a pharmaceutical composition comprising an autophagy modulating agent selected from formula (I):

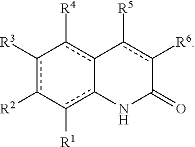

In some embodiments, R¹, R², R³, R⁴, R⁵, or R⁶ is hydrogen (H), halo (e.g., Cl, F), C₁₋₈alkyl (e.g., methyl, ethyl, butyl, propyl, isopropyl, isopentyl), halogen-substituted C₁₋₈alkyl (e.g., trifluoromethyl), piperidinyl (e.g., piperidin-1, 2, 3, or 4-yl), C₃₋₁₀cycloalkyl (e.g., phenyl), halogen-substituted C₃₋₁₀cycloalkyl (e.g., chlorophenyl), pyrimidinyl (e.g., pyrimidin-2,4, or 5, -yl), C₁₋₈alkoxy (e.g., methoxy, ethoxy, propoxy, butoxy, hexoxy), halogen substituted C₁₋₈alkyl (e.g., trifluoromethyl, trifluorobutoxy, trifluoropentoxy), C₁₋₆alkyl-C₃₋₁₀cycloalkoxy (e.g., cyclopropylethoxy), C₃₋₁₀cycloalkyl-C₁₋₆alkyl-amino (e.g., cyclopropylethylamino, cyclopropylethyl(methyl)amino), C₃₋₁₀heterocyclyloxy (e.g., piperidinyloxy, cyclopentylpiperidinyloxy), C₁₋₈alkylsulphonyl (e.g., methyl sulfonyl), C_{1 -8}alkylsulphonylalkoxy (e.g., methyl sulfonylpropoxy), C₁₋₁₀cycloalkoxy (e.g., phenyl propoxy), alkoxyC₁₋₁₀cycloalkyl (e.g., methoxy phenyl), substituted phenyl; and/or
R¹ or R², R² or R³, or R³ or R⁴ optionally form a ring (e.g., heterocyclyl, cycloalkyl, furanyl, pyridinyl, phenyl, etc.); a 5 or 6 membered ring (e.g., pyridinyl, furanyl, cycloalkyl, heterocyclyl); a substituted 5- or 6-membered ring (e.g., an alkyl-, alkyl-phenyl-, hydroxyl-, halo-, chloro-, bromo-, methoxy-, di-methoxy-, oxy-, or benzyl-substituted 5 or 6 membered ring); or a substituted bicyclic group (e.g., indole, azobicyclo, bridged bicyclic).

In some embodiments, the autophagy modulating agent is selected from:
(i) a 5,6-disubstituted quinolone;

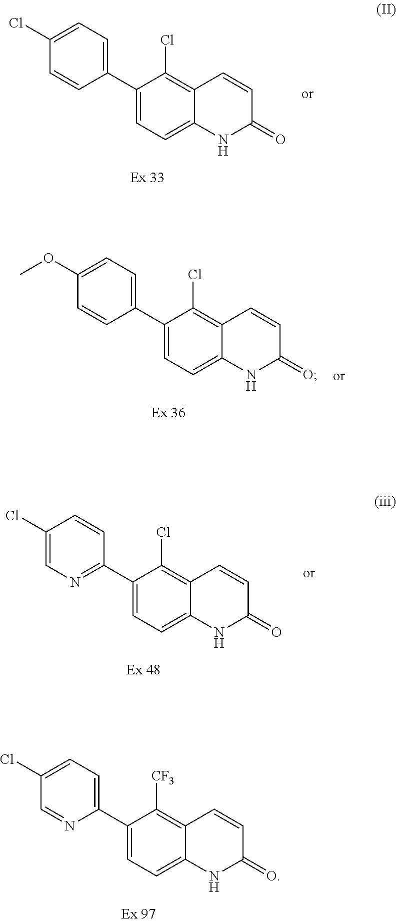 [SMILES: O=c1ccc2c(Cl)c(-c3ccc(Cl)cc3)ccc2[nH]1 ; COc1ccc(-c2ccc3[nH]c(=O)ccc3c2Cl)cc1 ; O=c1ccc2c(Cl)c(-c3ccc(Cl)cn3)ccc2[nH]1 ; O=c1ccc2c(C(F)(F)F)c(-c3ccc(Cl)cn3)ccc2[nH]1]

In some embodiments, the autophagy modulating agent is

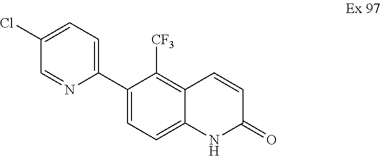 [SMILES: O=c1ccc2c(C(F)(F)F)c(-c3ccc(Cl)cn3)ccc2[nH]1]

(6-(5-chloropyridin-2-yl)-5-(trifluoromethyl) quinolin-2 (1H)-one).

In some embodiments, the autophagy modulating agent of formula (I) is selected from the group consisting of:

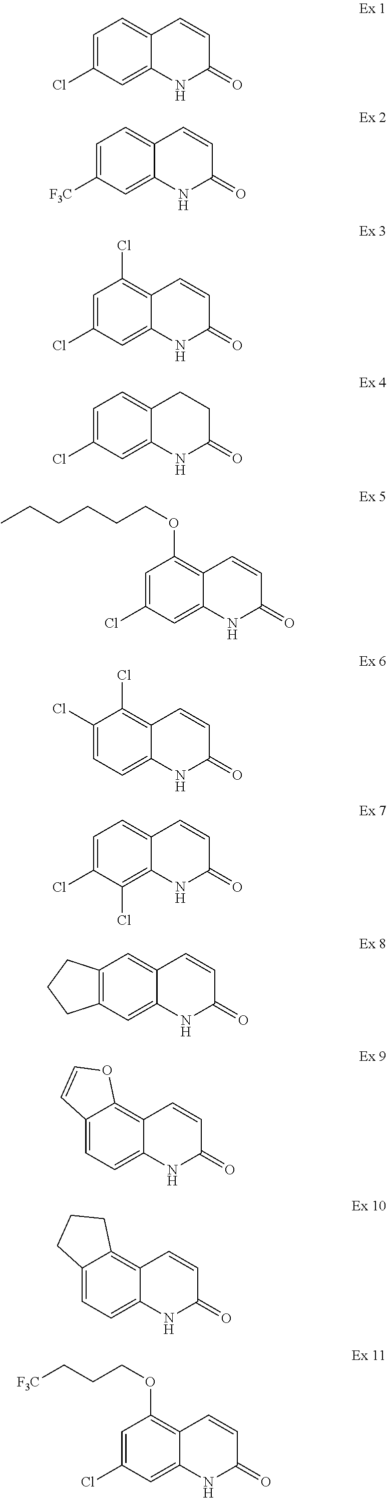

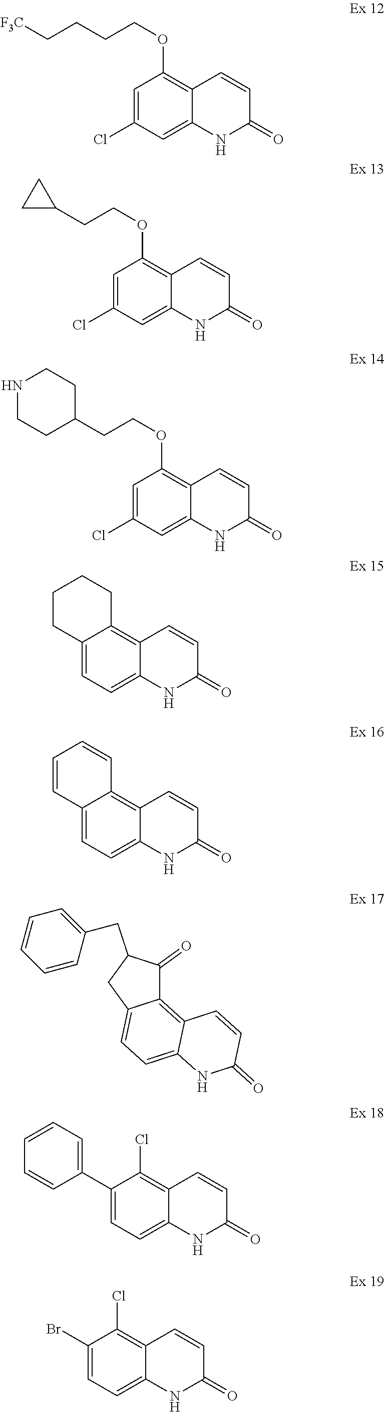

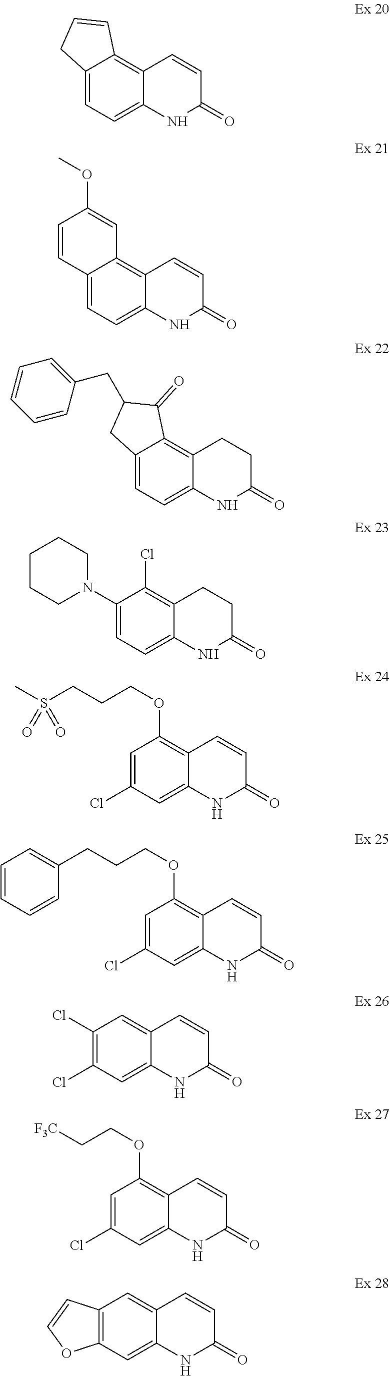

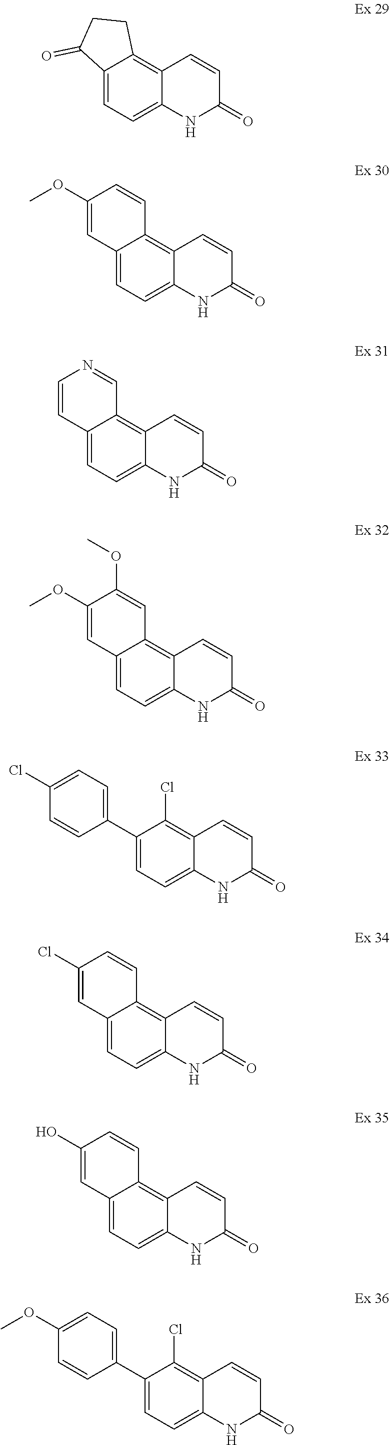

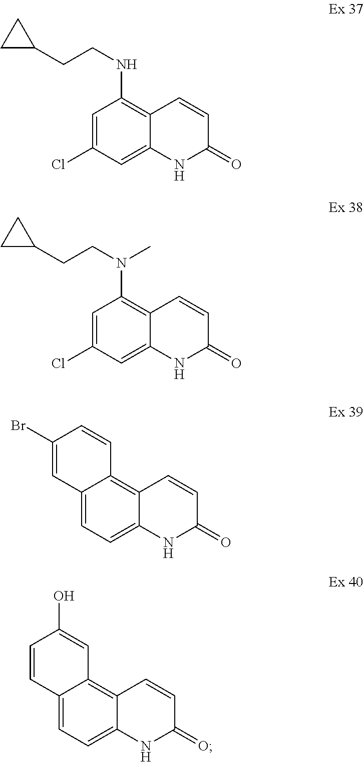 [SMILES: O=c1ccc2c(NCCC3CC3)cc(Cl)cc2[nH]1 ; CN(CCC1CC1)c1cc(Cl)cc2[nH]c(=O)ccc12 ; O=c1ccc2c(ccc3cc(Br)ccc32)[nH]1 ; O=c1ccc2c(ccc3ccc(O)cc32)[nH]1]

or
a pharmaceutically acceptable salt thereof, including all tautomers or stereoisomers, or optionally substituted analogs thereof.

In some embodiments, the autophagy modulating agent of formula (1) is selected from the group consisting of:

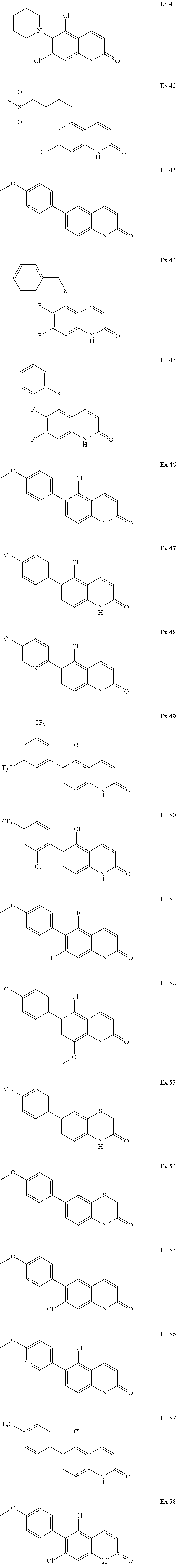

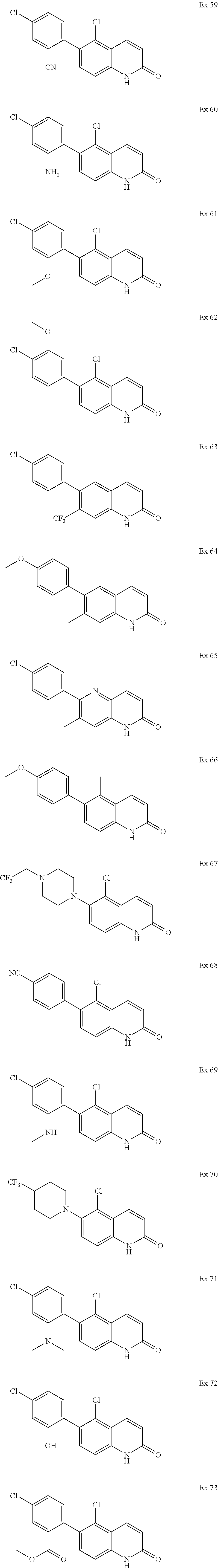

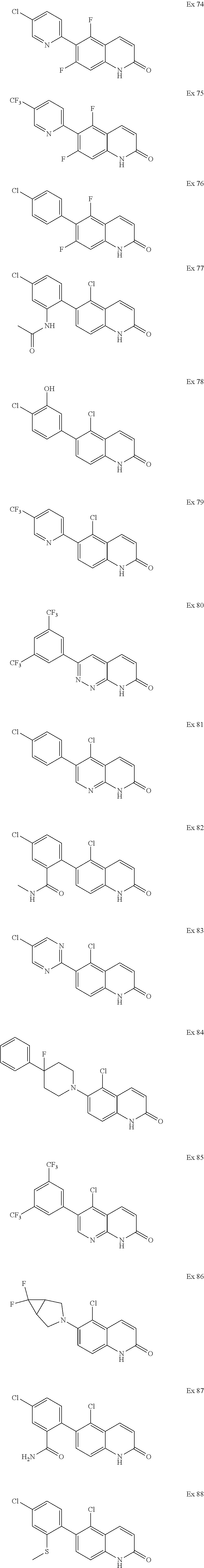

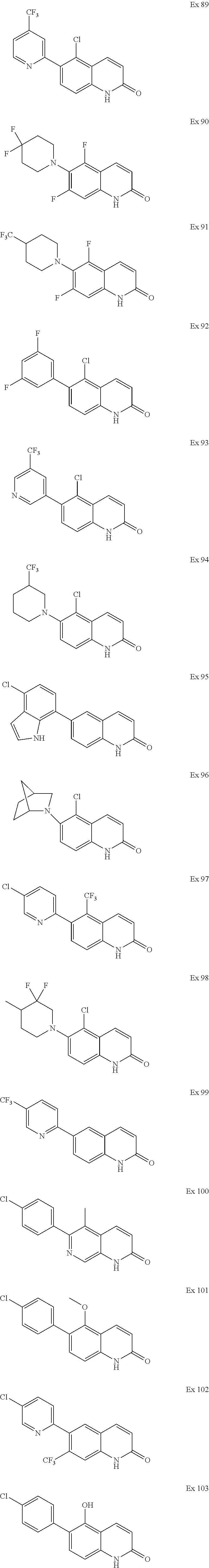

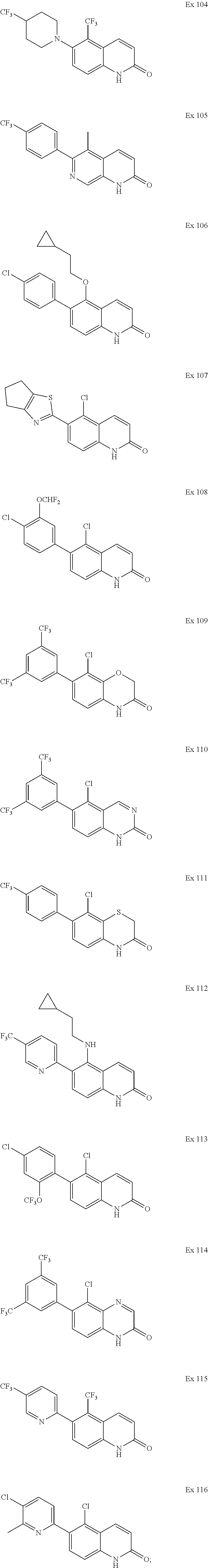

or
a pharmaceutically acceptable salt thereof, including all tautomers and stereoisomers, and optionally substituted analogs thereof.

In some embodiments, R¹ is hydrogen.

In some embodiments, the autophagy modulating agent has autophagy modulating activity. In some embodiments, the autophagy modulating agent is an autophagy enhancing agent or an autophagic pathway modulating agent. In some embodiments, the subject has or is suspected of having an autophagy-associated disease, disorder, or condition. In some embodiments, the autophagy-associated disease, disorder, or condition is alpha-1 antitrypsin deficiency (ATD). In some embodiments, the autophagy-associated disease, disorder, or condition is liver disease from alpha-1-antitrypsin deficiency (ATD). In some embodiments, the autophagy-associated disease, disorder, or condition is a polyglutamine (polyQ) disease. In some embodiments, the polyQ disease is Huntington's disease (HD); spinocerebellar ataxias (SCA) types 1, 2, 6, 7, 17; Machado-Joseph disease (MJD/SCA3); dentatorubral pallidoluysian atrophy (DRPLA); spinal and bulbar muscular atrophy; or X-linked 1 (SMAX1/SBMA). In some embodiments, the autophagy-associated disease, disorder, or condition is Alzheimer's disease (AD). In some embodiments, the autophagy-associated disease, disorder, or condition is inherited emphysema. In some embodiments, the autophagy-associated disease, disorder, or condition is diabetes. In some embodiments, the autophagy-associated disease, disorder, or condition is Huntington's disease (HD). In some embodiments, the autophagy-associated disease, disorder, or condition is cancer. In some embodiments, the autophagy-associated disease, disorder, or condition is an age-dependent degenerative disease. In some embodiments, the therapeutically effective amount of the pharmaceutical composition comprising the autophagy modulating agent reduces aggregated ATZ protein in the subject having α1-antitrypsin deficiency (ATD). In some embodiments, the therapeutically effective amount of the pharmaceutical composition comprising the autophagy modulating agent reduces cellular accumulation of misfolded aggregated α1-antitrypsin Z variant (ATZ). In some embodiments, the therapeutically effective amount of the pharmaceutical composition comprising the autophagy modulating agent reduces cellular accumulation of misfolded or aggregated proteins. In some embodiments, the therapeutically effective amount of the pharmaceutical composition comprising the autophagy modulating agent has anti-tumor activity in the subject having cancer. In some embodiments, the therapeutically effective amount of the pharmaceutical composition comprising the autophagy modulating agent reduces neuronal cell death. In some embodiments, the therapeutically effective amount of the pharmaceutical composition comprising the autophagy modulating agent treats or prevents hepatic fibrosis. In some embodiments, the therapeutically effective amount of the pharmaceutical composition comprising the autophagy modulating agent reduces or prevents accumulation of misfolded protein in liver cells, liver damage, liver fibrosis, or liver failure. In some embodiments, the therapeutically effective amount of the pharmaceutical composition comprising the autophagy modulating agent reduces liver fibrosis. In some embodiments, the therapeutically effective amount of the pharmaceutical composition comprising the autophagy modulating agent inhibits the progression of the autophagy-associated disease, disorder, or condition. In some embodiments, the therapeutically effective amount of the pharmaceutical composition comprising the autophagy modulating agent does not significantly affect insulin secretion.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1E is an exemplary embodiment showing autophagy activator rescues HD-MSNs from degeneration in accordance with the present disclosure. FIG. 1A depicts the chemical structure of Ex 3. FIG. 1B includes a representative immunoblot (left) for steady state levels of α1-antitrypsin Z variant (ATZ) and β-actin in the HTO/Z cell line model of α1-antitrypsin deficiency after 24 hr treatment with DMSO alone or Ex 3 in DMSO at 0.5 and 2.5 μM and a bar graph showing the normalized intensity (right) calculated from immunoblot images for 0.5 μM versus DMSO control (Individual data plotted, n=4 biological replicates; *p=0.003 by t-test; mean+/−s.e.m). FIG. 1C includes an immunoblot for p62 and GAPDH in HD-MSNs treated with DMSO or 0.5 μM Ex 3 at PID26 (left) and a bar graph showing intensity values were normalized by GAPDH intensities and the relative fold change over DMSO condition was calculated from immunoblot images of three independent HD-MSN lines (right). Each dot represents reprogrammed MSNs from three independent HD-MSN lines. FIG. 1D includes images and a bar graph showing measurements of autophagic vacuoles by CYTO-ID green from four independent HD-MSN lines treated with DMSO or 0.5 μM Ex 3 at PID 26. Individual dots represent 12 biological replicates. FIG. 1E includes example photographs (top) of HD-MSNs treated with DMSO or 0.5 μM Ex 3 (PID 30) expressing the tandem monomeric RFP-GFP-LC3 reporter and a bar graph (bottom) showing quantification of autophagosome (i.e., RFP+, GFP+ puncta) and autolysosome (i.e., RFP+, GFP− puncta) compartments from three independent HD-MSNs. Measurements were performed in cells having at least 3 puncta per cell (from more than 50 cells per MSN line). FIG. 1F includes representative images of SYTOX staining (top) and a graph showing quantification (bottom) of SYTOX-positive cells as a fraction of Hoechst-positive cells in HD-MSN (n=4). FIG. 1G includes representative images (left) and graphs showing quantification (right) of caspase3/7 activation (green) and Annexin V signal (green) in HD-MSN treated with DMSO or different concentration of Ex 3 (0.125, 0.25, or 0.5 uM) (n=4-8). FIG. 1H includes graphs showing quantification of caspase activation (top) and Annexin V signal (bottom) from four independent HD-MSN lines treated with DMSO or 0.5 uM Ex 3. Individual dots represent 9 biological replicates. FIG. 1I includes representative images (top) and a bar graph showing quantification (bottom) of HTT inclusion bodies (IBs) from three independent HD-MSNs treated with DMSO or 0.5 μM Ex 3 (DMSO: 285 cells, 0.5 μM Ex 3:325 cells) at PID30. Each dot represents one individual's reprogrammed HD-MSN. For all figures shown, statistical significance was determined using unpaired t-test; ****p<0.0001, ***p<0.001, **p<0.01, *p<0.05. Scale bars in FIG. 1D 10 μm; in FIG. 1E, FIG. 1F, and FIG. 1I 20 μm; in FIG. 1G 100 μm. Mean±s.e.m.

FIG. 2 shows a synthetic route for the preparation of Ex 3 in accordance with the present disclosure.

FIG. 3 shows a synthetic route for the preparation of Ex 97 in accordance with the present disclosure.

FIG. 4A-FIG. 4B are graphs showing the results of an In-Cell-Western assay depicting inhibition of poly ATZ in HTOZ cells by various quinolone compounds of the present disclosure.

FIG. 5A-FIG. 5B are western blot images showing the effect of GLB analogs of the present disclosure on ATZ levels in HTO/Z cell line engineered to disrupt macro-autophagy (STX17KO).

FIG. 6 is a western blot image showing the effect of GLB analogs of the present disclosure on ATZ levels in HTO/Z cell line engineered to disrupt macro-autophagy (ATG14KO).

FIG. 7 shows the effect of Ex 97 on steady state levels of ATZ in HeLaTO/Z in accordance with the present disclosure.

### DETAILED DESCRIPTION OF THE INVENTION

The present disclosure is based in part on the discovery of autophagy modulating agents for the modulation (i.e., enhancement) of autophagy; the treatment of alpha-1-antitrypsin deficiency (ATD); the treatment of Huntington's disease (HD); and other autophagy-associated diseases. A new series of analogs described herein have increased potency and stability.

ATD is an inherited disorder that can result in liver disease. As described herein, a series of glyburide (e.g., glibenclamide or GLB) structural analogs have also been created that exhibited improved efficacy compared to glyburide.

Taken together these results are consistent with an effect of the analog on two different types of misfolded proteins and two different types of misfolded diseases, as would be predicted for an autophagy enhancer drug. The data also suggests that these analogs and derivatives could be therapeutic for other misfolded protein diseases and age-dependent degenerative diseases, as well.

### Autophagy-Associated Diseases, Disorders, or Conditions

Another aspect of the present disclosure provides for modulation of autophagy in a subject suffering from an autophagy-associated disease, disorder, or condition. Autophagy can be described as a pathway or process in a cell or organism that involves degradation of cellular components via delivery to a lysosome. For example, the pathway can be chaperone-mediated autophagy, microautophagy, macroautophagy, or variants of macroautophagy, such as LC3-associated phagocytosis. An autophagy-associated disease, disorder, or condition may also be one that involves membrane trafficking pathways that are dependent on autophagy genes, such as ‘unconventional protein secretion’, ‘exocytosis of secretory granules/lysosomes’, ‘exosome secretion’, or ‘retromer-dependent trafficking’. These pathways are capable of removing misfolded proteins, aggregated proteins, or parts of organelles by delivery to the plasma membrane for exocytosis without involving degradation and/or delivery to the lysosome. As such, an autophagy modulating agent can be capable of modulating such autophagic pathways.

As described herein, an autophagy-associated disease, disorder, or condition can be a disease resulting from defects in, or abnormal function of, autophagic processes or autophagic pathways in a cell or organism; diseases and disorders that are caused by misfolded and/or aggregated proteins, which can include age-dependent degenerative diseases; or diseases in which autophagy function has been implicated. Defects in, or abnormal function of, an autophagic pathway or process may involve a defect in, or abnormal function of the action of various cellular components, such as an organelle or protein. For example, the organelle can be a lysosome, a vesicle, an autophagosome, a vacuole, a phagophore, or a plasma membrane. As another example, the protein can be insulin, insulin growth factor, insulin receptor, a TOR or mTOR protein, an Atg protein, Ras, PKA, Sch9, Gcn2, elF2alpha, Gcn4, Snf1, Pho85, PDK1, PTEN, Rheb, TSC1, TSC2, AMPK, Beclin1, Bcl-2, LKB1, p70S6K, p27, EF1 alpha, GFAP, LAMP-2A, Hsp90, hsc70, aldolase B, Annexin, aspartate aminotransferase, Fos, Eps8, hemoglobin, Pax2, MEF2D, microglobulin, phosphoglycerate mutase, pyruvate kinase, RCAN1, RNAse A, alpha synuclein, subunits of 20S proteasome, tau, or ubiquitin. As such, an autophagy-associated disease, disorder, or condition can be a disease, disorder, or condition associated with the autophagy pathway or dysfunction in the above organelles or proteins among others. As such, an autophagy modulating agent can be capable of modulating such autophagic pathways.

For example, an autophagy modulating agent described herein and analogs thereof can be used as a therapeutic for ATD, Huntington's, and other misfolded protein and age-dependent degenerative diseases in which enhancing autophagy will be beneficial.

For example, an autophagy-associated disease, disorder, or condition can be adult polyglucosan body disease, Afibrinogenemia, alpha-1 antitrypsin deficiency (ATD), Alzheimer's disease (AD), amyotrophic lateral sclerosis, an age-dependent degenerative disease, autism spectrum disorders, Becker muscular dystrophy, beta-propellar protein-associated neurodegeneration, Birt-Hogg-Dube syndrome, Blau syndrome, cancer, Centronuclear myopathy, Chanarin-Dorfman syndrome, Charcot-Marie-Tooth (CMT) disease, childhood ataxia, Chorea-acanthocytosis, Chronic progressive external ophthalmoplegia, Congenital disorders of glycosylation, Congenital dyserythropoietic anemia, Congenital myasthenic syndrome, Congenital myotonic dystrophy, Corneal dystrophy Avellino type, cortical atrophy, Crohn's disease, Danon disease, Danon's cardiomyopathy, diabetes, distal myopathy, Dysferlinopathy, Emery-Dreifuss muscular dystrophy, epilepsy, Familial encephalopathy with neuroserpin inclusion bodies, familial Mediterranean fever, Familial partial lipodystrophy, Fanconi anemia congenital syndrome, frontotemporal dementia, Galloway-Mowat syndrome, Gaucher's disease, Gerstmann-Straussler-Scheinker disease, Glycogen storage disease type 2, Griscelli syndrome, Groenouw type I corneal dystrophy, Hermansky-Pudlak syndrome, Huntington's disease (HD), Idiopathic pulmonary fibrosis, inflammatory bowel disease, juvenile arthritis, Kearns-Sayre syndrome, Keshan disease, LEOPARD syndrome, Li-Fraumeni syndrome, Limb-girdle muscular dystrophy type 1D, 2B, LRBA deficiency, Macrophagic myofasciitis, Marek disease, Martsolf syndrome, Miyoshi myopathy, Mulibrey Nanism, multiple sclerosis (MS), multisystem disorder, cystinosis, Myofibrillar myopathy, Myostatin-related muscle hypertrophy, Myotonic dystrophy, Nemaline myopathy, neuronal ceroid lipofuscinosis, Neuronal ceroid lipofuscinosis, non-alcoholic fatty liver disease, NORSE, osteoarthritis, osteopetrosis, Paget's disease of the bone, Papillon Lefevre syndrome, Parkinson's disease, Pelger-Huet anomaly, Perry syndrome, Peters anomaly, Phosphoglycerate kinase deficiency, primary microcephaly, primary open angle glaucoma. Progeria, Proteus syndrome, Reducing body myopathy, Retinitis pigmentosa, Rett syndrome, Salla disease, SAPHO syndrome, Schaaf-Yang syndrome, Sengers syndrome, sensory and autonomic neuropathy type II, SHORT syndrome, Simpson-Golabi-Behmel syndrome, Sitosterolemia, Smith-Magenis syndrome, Snyder-Robinson syndrome, spastic paraplegia, spinocerebellar ataxia, Stargardt disease, systematic lupus erythematosus, systemic sclerosis, Tangier disease, tuberculosis, ulcerative colitis, Vici syndrome, Wiskott Aldrich syndrome, X-linked myopathy with excessive autophagy, X-linked myotubular myopathy, Yunis-Varon syndrome, or Zellweger syndrome spectrum disorders.

As another example, an autophagy-associated disease, disorder, or condition can be diseases and disorders that are caused by misfolded and/or aggregated proteins, which can include age-dependent degenerative diseases, Amyotonia congenita, Benign hereditary chorea, Bethlem myopathy, Bourneville syndrome, Brown syndrome, Central diabetes insipidus, Charcot-Marie-Tooth disease, Cholesteryl ester storage disease, Chorea minor, Cramp-fasciculation syndrome, Dentatorubral-pallidoluysian atrophy, Doxorubicin-induced cardiomyopathy, Episodic ataxia with nystagmus, Fabry disease, Familial Mediterranean fever, Froster-Huch syndrome, Hypergonadotropic ovarian failure, familial or sporadic, Idiopathic inflammatory myopathy, Inclusion body myositis, Kennedy disease, Lafora disease, Leber congenital amaurosis 11, Leber congenital amaurosis 3, Limb-girdle muscular dystrophy, Marinesco-Sjogren syndrome, Oculopharyngeal muscular dystrophy, Pancreatitis, pediatric, Pelizaeus-Merzbacher disease. Phenylketonuria, Pigment-dispersion syndrome, Refsum disease, infantile form, Spinal muscular atrophy, Spinocerebellar ataxia, or Tubular aggregate myopathy.

As another example, an autophagy-associated disease, disorder, or condition can be a disease resulting from defects in, or abnormal function of, autophagic processes or autophagic pathways in a cell or organism or diseases in which autophagy function has been implicated such as adult polyglucosan body disease, Afibrinogenemia, Centronuclear myopathy, Congenital dyserythropoietic anemia, Congenital myotonic dystrophy, Danon disease, Familial encephalopathy with neuroserpin inclusion bodies, Hermansky-Pudlak syndrome, Idiopathic pulmonary fibrosis, Miyoshi myopathy, Myofibrillar myopathy, Myotonic dystrophy, Progeria, Retinitis pigmentosa, Stargardt disease, X-linked myopathy with excessive autophagy, or X-linked myotubular myopathy.

### Alpha-1 Antitrypsin Deficiency (ATD)

An aspect of the present disclosure provides for modulation of autophagy in a subject suffering from alpha-1 antitrypsin deficiency (ATD). ATD is an inherited disorder that can result in liver disease, due to accumulation of misfolded mutant alpha-1 antitrypsin protein (ATZ). ATD is a well-known genetic cause of severe liver disease including cirrhosis and hepatocellular carcinoma in adults. The classical form of ATD is characterized by a point mutation that substitutes lysine for glutamate 342 in the mutant variant called ATZ.

The inventors have discovered that glibenclamide (GLB), an FDA approved drug for type 2 diabetes, enhances the autophagic degradation of misfolded ATZ and therefore is a potential therapeutic for ATD. In those studies they proved that the mechanism of action of GLB was enhanced autophagic degradation of ATZ in a number of ways but most definitively by showing that the drug effect was blocked when the autophagy gene ATG14 was deleted in a mammalian cell line model of ATD. Furthermore, an analog (“G2”) of the parent drug decreased hepatic ATZ load together with increased LC3-II conversion and decreased p62 levels in the liver of the PiZ mouse model of ATD, markers of increased autophagic activity. As such, a series of GLB analogs were created (see Ex 1-116). Analogs show reduced hepatic ATZ load and fibrosis in a PiZ mouse model of ATD without affecting insulin secretion. These analogs also decreased cellular ATZ load in the C. elegans model of ATD in a dose-dependent fashion.

GLB analogs also improved the survival of human striatal neurons derived from patients with Huntington's disease and also lowers HTT inclusion body (IB) in HD-MSNs in contrast to the inactive form.

These results are consistent with an effect of GLB analogs on two different types of misfolded proteins and two different types of misfolded diseases, as would be consistent with an effect on autophagic degradation by an autophagy enhancing agent or drug. This data, together with the known functions of autophagy, suggests that GLB analogs or derivatives can be therapeutic or preventative for misfolded protein diseases and age-dependent degenerative diseases.

New optimized analogs were developed and described herein, such as Ex 3. Treatment using Ex 3, increased the resilience of HD-MSNs against neuronal death by promoting autophagy towards the pre-HD-MSN state, demonstrating that the autophagic decline during aging in HD underlies MSN degeneration and pointing to potential approaches for enhancing autophagy and resilience of MSNs against degeneration in HD. The new GLB analog Ex 3, designed to increase the potency of the compound, decreased the steady-state levels of ATZ in a HTO/Z cell line model of ATD. Treating HD-MSNs with Ex 3 decreased p62/SQSTM1 protein levels and increased the number of autophagic vacuoles as measured by CYTO-ID signals. Also, Ex 3 increased the average number of autophagosomes and autolysosomes in HD-MSNs expressing the RFP-GFP-LC3 reporter, verifying the autophagy-enhancing activity of Ex 3 in HD-MSNs. Cells were treated with Ex 3 at PID14, a time point when cells undergoing miRNA-mediated conversion adopt neuronal identity, and further treated every four days for 16 days. Importantly, Ex 3 resulted in a significant reduction in HD-MSN death in a dose-dependent manner, as measured by the SYTOX assay and in activated Caspase 3/7 and Annexin V signals in HD-MSNs at PID30. This result was confirmed in three other independent lines of HD-MSNs. Ex 3 also reduced the number of mHTT inclusion bodies in HD-MSNs compared to DMSO treatment. Together, the data demonstrate that enhancing autophagy in HD-MSNs alleviates the degenerative state. These findings are interesting because in mouse embryonic fibroblasts harboring the repeat number in the range of 100, mutant HTT was shown to fail to be loaded into autophagosomes. The results described herein, however, challenge this notion such that in neurons derived from adult-onset patients, enhancing autophagy increases HD-MSNs' ability to clear mHTT aggregation and their resilience against neurodegeneration.

The classical form of ATD is characterized by a point mutation that substitutes lysine for glutamate 342 in the mutant variant called ATZ. The substitution is known to favor misfolding of ATZ and sets up a kinetic-determined tendency for this variant protein to polymerize and form aggregates in the endoplasmic reticulum (ER) and perhaps other pre-Golgi vesicular compartments of the cell. There is evidence that liver disease is caused by gain-of-function mechanisms triggered by the proteotoxic effects of misfolded ATZ accumulation. Genetic and environmental modifiers that target proteostasis mechanisms are hypothesized to account for wide variation in the hepatic phenotype among homozygotes for this disorder.

### ATD Disease Models

Transgenic C. elegans (‘Z worm’) expressing the human Z mutant form of alpha-1 antitrypsin (ATZ) fused to green fluorescent protein (GFP) can be used as a model of ATD for screening and testing of autophagy modulating agents to treat ATD. The C. elegans model of ATD exhibits ATZ aggregation within the endoplasmic reticulum, slow growth, reduced fertility, and shortened lifespan. These phenotypes are also exhibited in humans with ATD, proving that C. elegans is a representative model of the disease.

A transgenic mouse model expressing the human mutant variant of alpha-1 antitrypsin, referred to as PiZ mouse, can also be used as a model of ATD. The PiZ mouse exhibits accumulation of mutant alpha-1 antitrypsin aggregates, liver fibrosis, and development of malignant liver tumors.

### Autophagy Modulating Agents

Another aspect of the present disclosure provides for modulation of autophagy in a subject, comprising administering to the subject a therapeutically effective amount of a composition comprising an autophagy modulating agent.

In some aspects of the present disclosure, the autophagy modulating agent can be of formula (I):

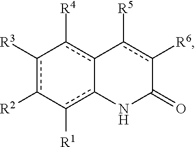

wherein R¹, R², R³, R⁴, R⁵, or R⁶ is hydrogen (H), halo (e.g., Cl, F), C₁₋₈alkyl (e.g., methyl, ethyl, butyl, propyl, isopropyl, isopentyl), halogen-substituted C₁₋₈alkyl (e.g., trifluoromethyl), piperidinyl (e.g., piperidin-1, 2, 3, or 4-yl), C₃₋₁₀cycloalkyl (e.g., phenyl), halogen-substituted C₃₋₁₀cycloalkyl (e.g., chlorophenyl), pyrimidinyl (e.g., pyrimidin-2,4, or 5, -yl), C₁₋₈alkoxy (e.g., methoxy, ethoxy, propoxy, butoxy, hexoxy), halogen substituted C₁₋₈alkyl (e.g., trifluoromethyl, trifluorobutoxy, trifluoropentoxy), C₁₋₆alkyl-C₃₋₁₀cycloalkoxy (e.g., cyclopropylethoxy), C₃₋₁₀cycloalkyl-C₁₋₆alkyl-amino (e.g., cyclopropylethylamino, cyclopropylethyl(methyl)amino), C₃₋₁₀heterocyclyloxy (e.g., piperidinyloxy, cyclopentylpiperidinyloxy), C₁₋₈alkylsulphonyl (e.g., methyl sulfonyl), C₁₋₈alkylsulphonylalkoxy (e.g., methyl sulfonylpropoxy), C₁₋₁₀cycloalkoxy (e.g., phenyl propoxy), alkoxyC₁₋₁₀cycloalkyl (e.g., methoxy phenyl), substituted phenyl; and/or
R¹ or R², R² or R³, or R³ or R⁴ optionally form a ring (e.g., heterocyclyl, cycloalkyl, furanyl, pyridinyl, phenyl, etc.); a 5 or 6 membered ring (e.g., pyridinyl, furanyl, cycloalkyl, heterocyclyl); a substituted 5- or 6-membered ring (e.g., an alkyl-, alkyl-phenyl-, hydroxyl-, halo-, chloro-, bromo-, methoxy-, di-methoxy-, oxy-, or benzyl-substituted 5 or 6 membered ring); or a substituted bicyclic group (e.g., indole, azobicyclo, bridged bicyclic).

For example, the autophagy modulating agent can be one of the following formulas:

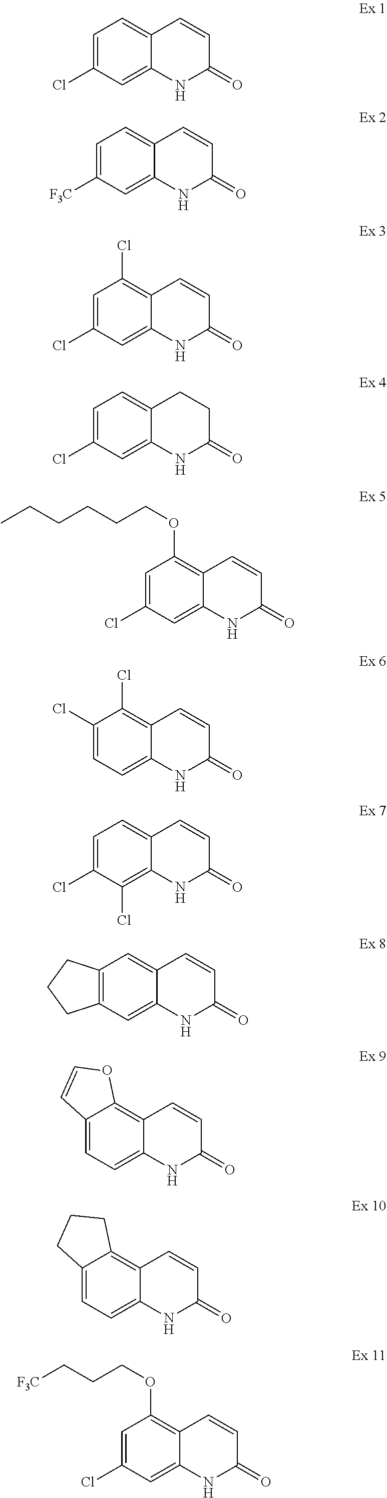

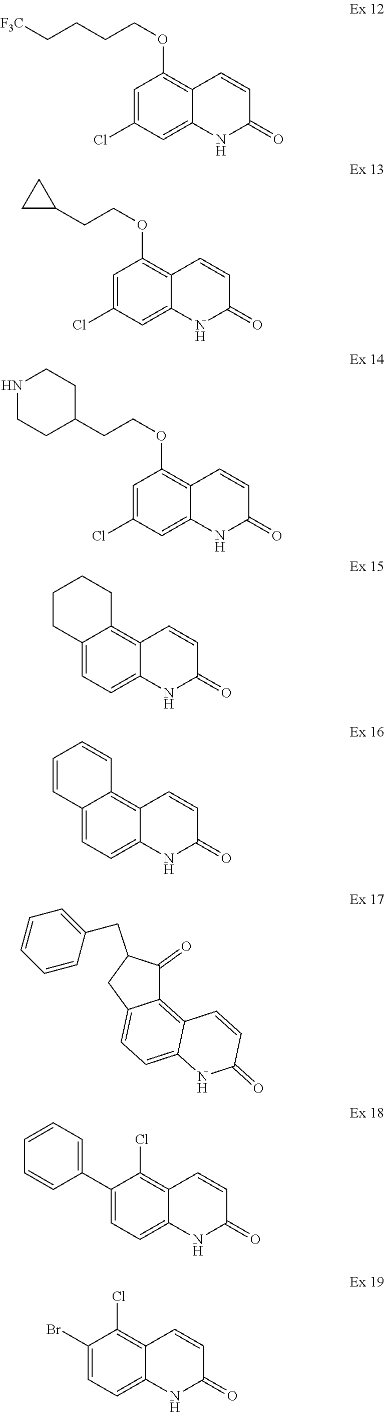

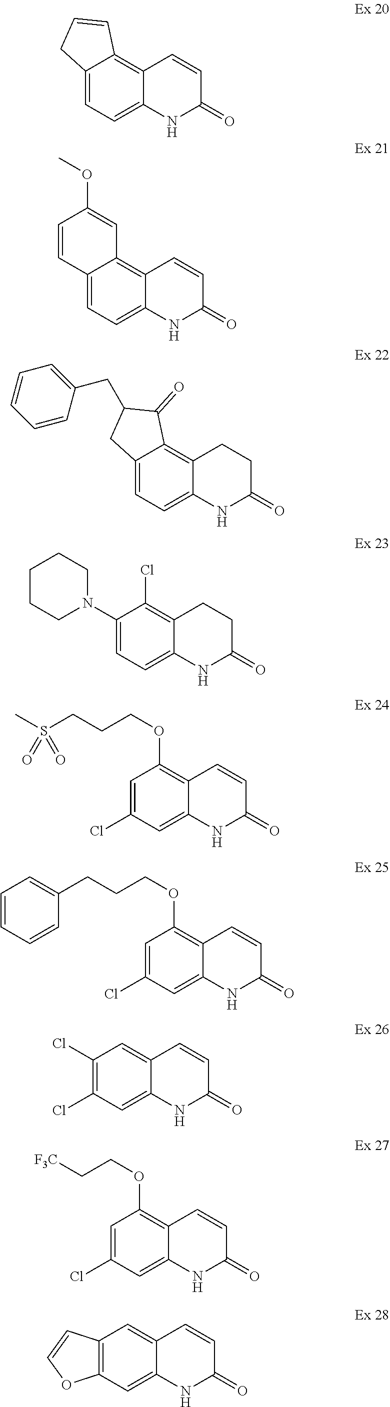

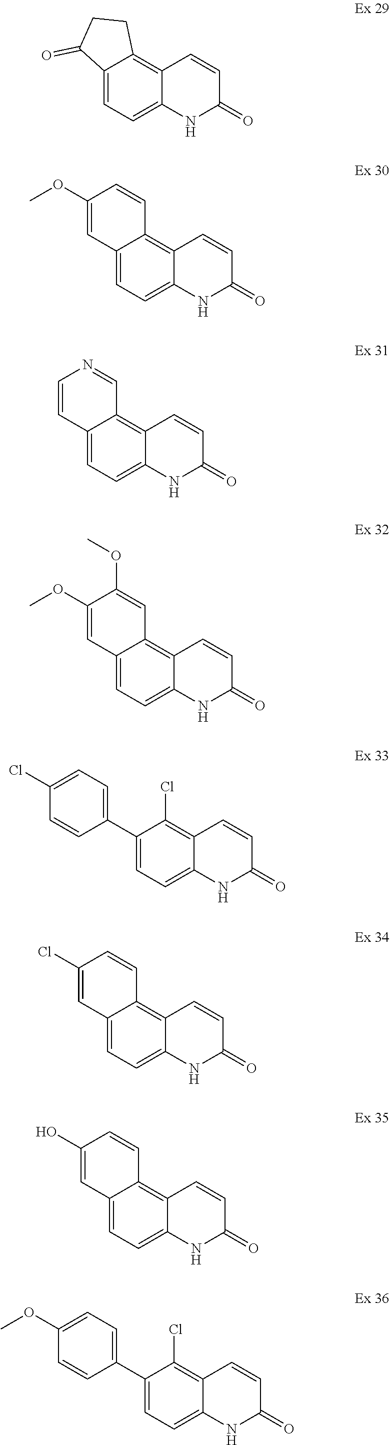

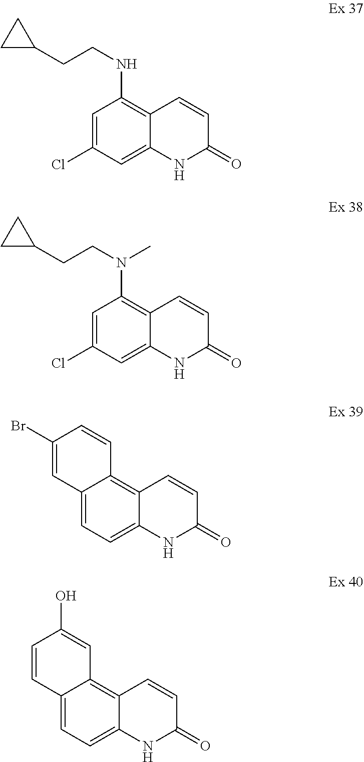 [SMILES: O=c1ccc2c(NCCC3CC3)cc(Cl)cc2[nH]1 ; CN(CCC1CC1)c1cc(Cl)cc2[nH]c(=O)ccc12 ; O=c1ccc2c(ccc3cc(Br)ccc32)[nH]1 ; O=c1ccc2c(ccc3ccc(O)cc32)[nH]1]

or pharmaceutically acceptable salts thereof, including all tautomers and stereoisomers, and substituted analogs thereof.

As another example, the autophagy modulating agent can be one of the following formulas:

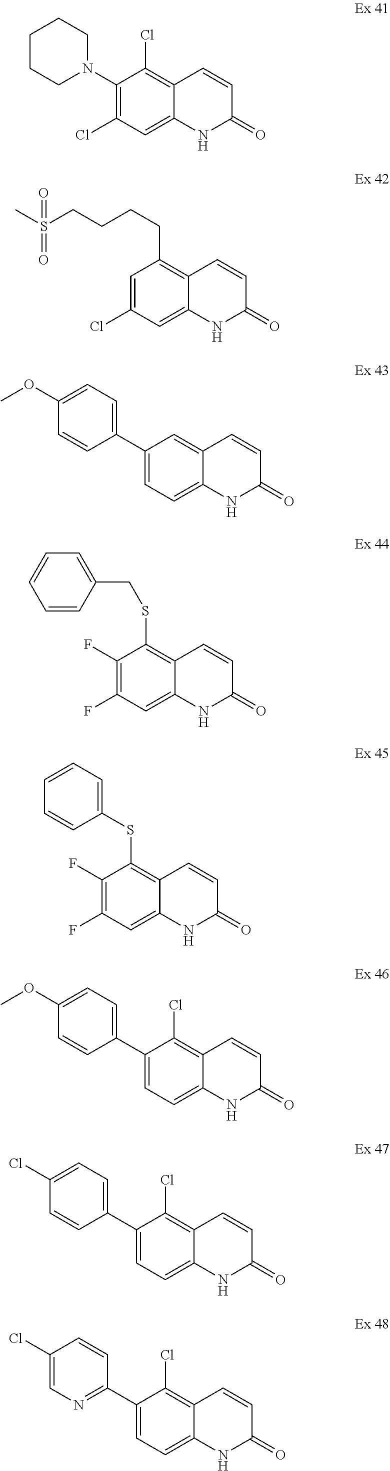

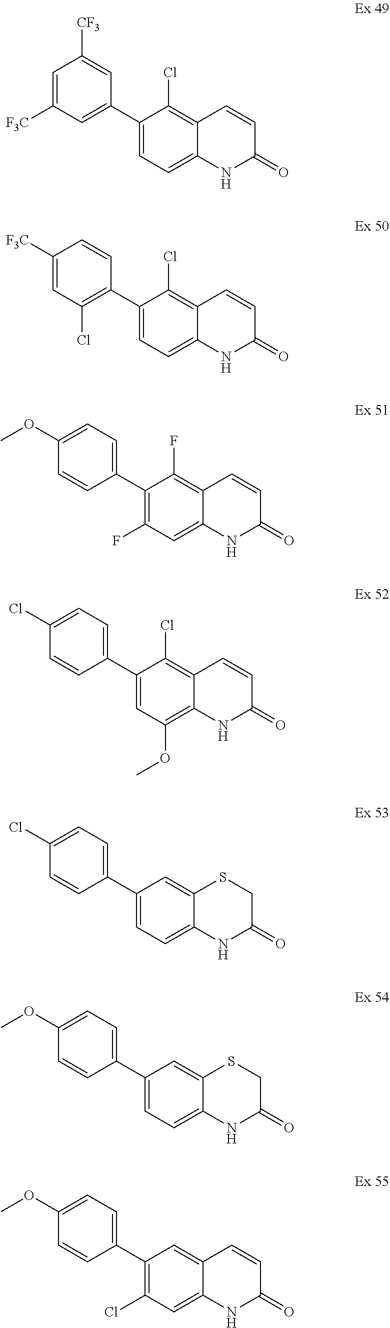

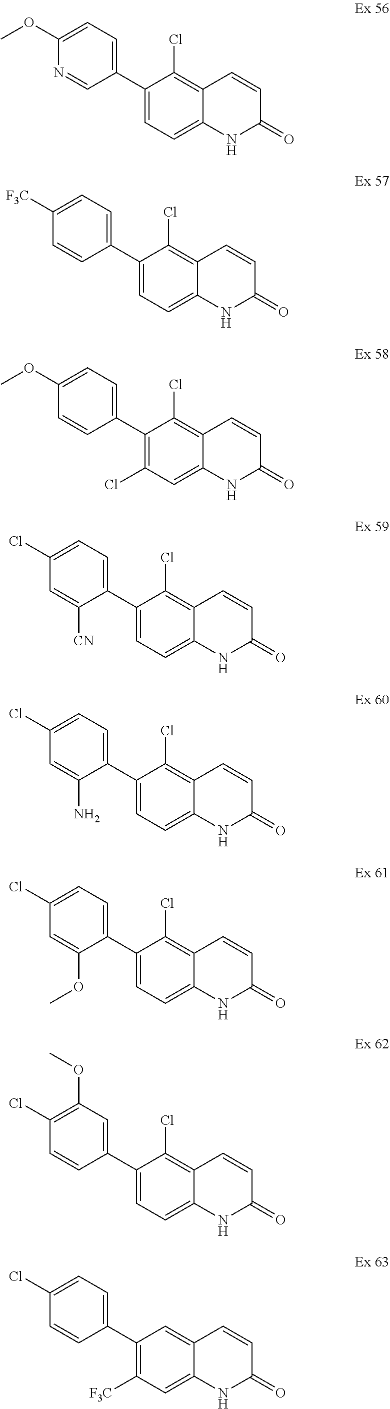

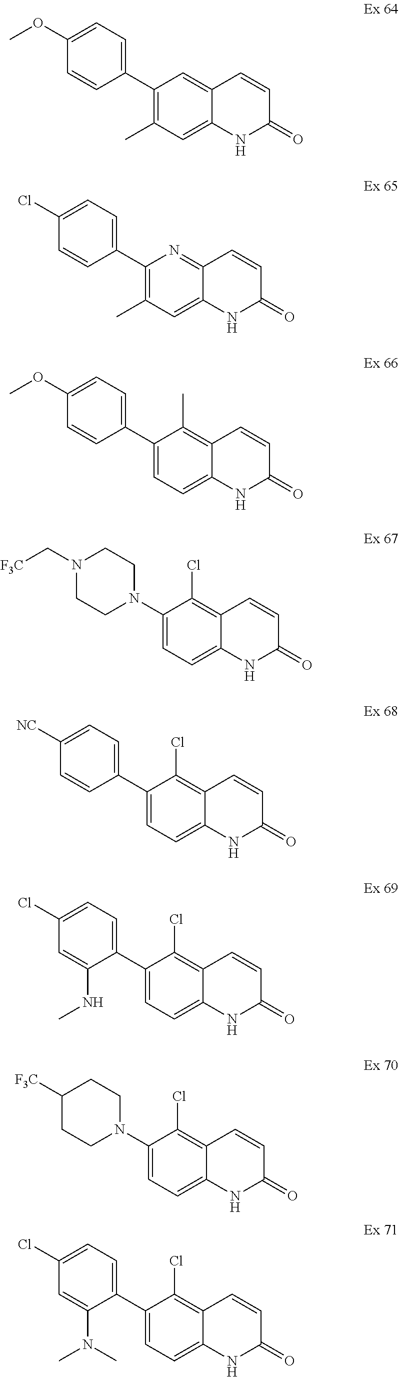

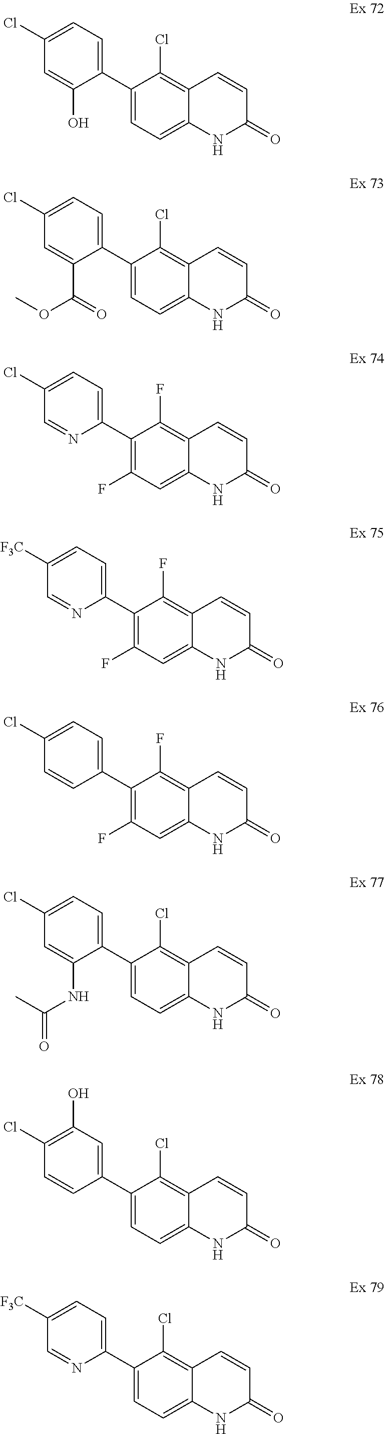

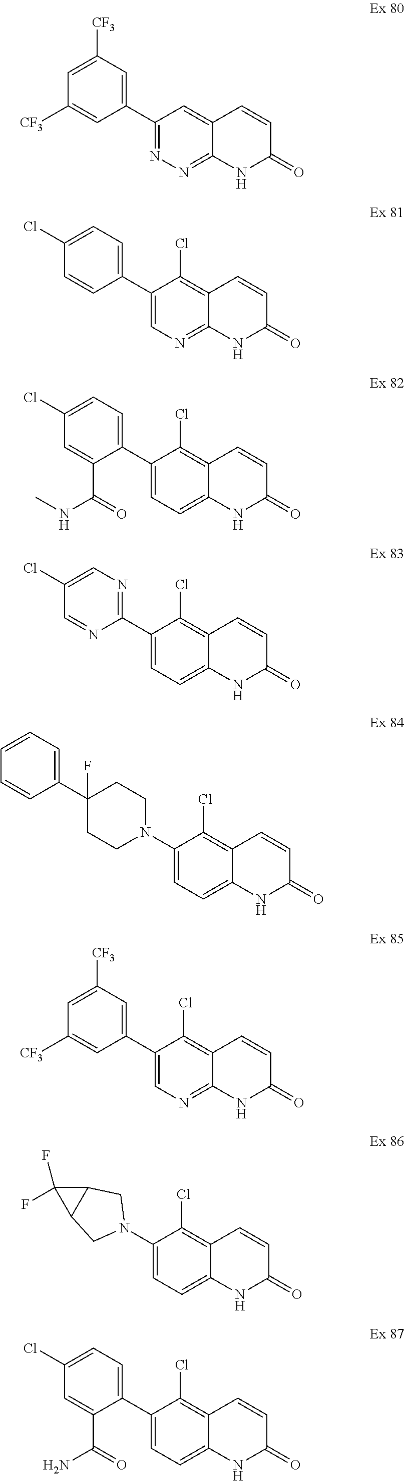

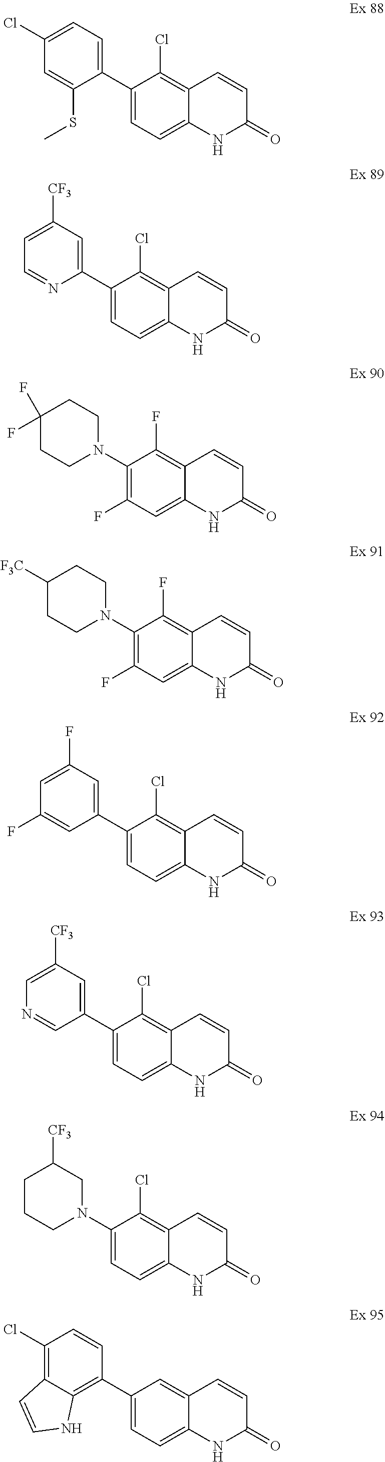

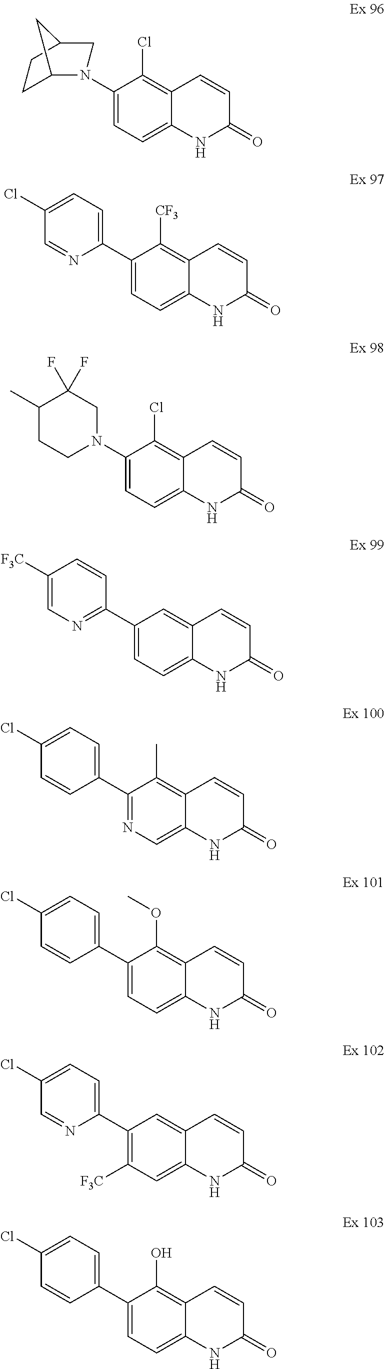

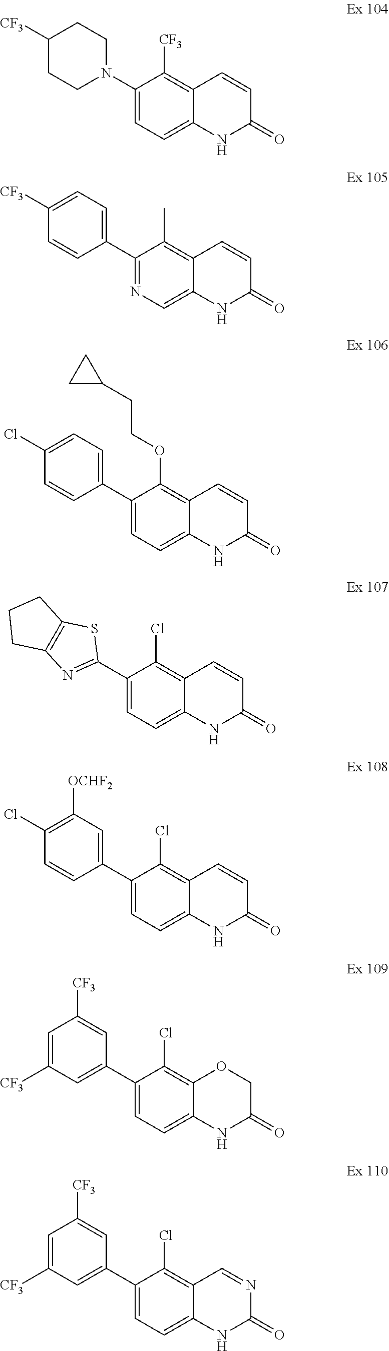

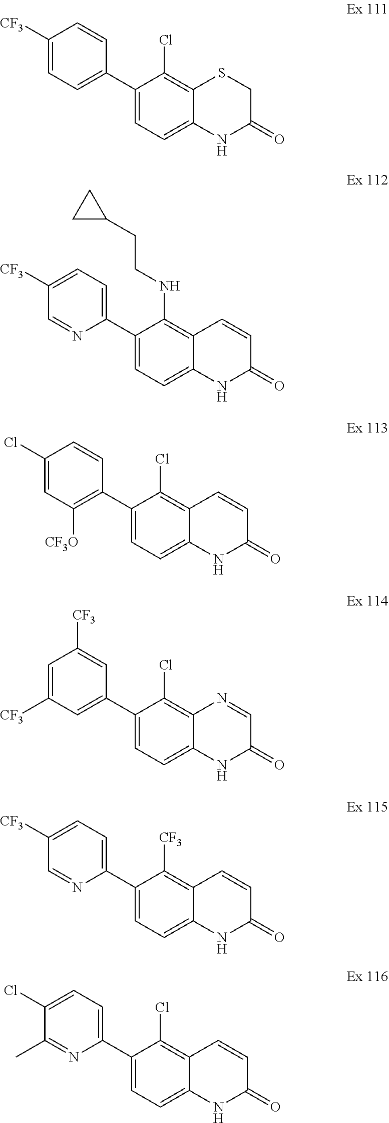

or pharmaceutically acceptable salts thereof, including all tautomers and stereoisomers, and substituted analogs thereof.

Any of the R groups (e.g., R¹, R², R³, R⁴, R⁵, or R⁶) (or a ring formed from the aforementioned R groups) can include or can be substituted or functionalized with hydrogen (H), amino, acetamide, cyano, halo (e.g., Cl, F, Br), C₁₋₈alkyl (e.g., methyl, ethyl, butyl, propyl, isopropyl, isopentyl), C₁₋₈alkoxy (e.g., methoxy), alkylamino (e.g., dimethylamino), C₃₋₁₀cycloamino (e.g., phenylamino), C₃₋₁₀heterocycloamino (e.g., pyridinylamino), halogen substituted C₁₋₈alkyl (e.g., trifluoromethyl), halogen substituted C₁₋₈alkoxy (e.g., OCF₃), C₃₋₁₀cycloalkyl (e.g., phenyl), C₁₋₁₀carbonyl, C₃₋₁₀cycloalkoxy (e.g., cyclopropoxy, alkylphenoxy, chlorophenoxy, benzyloxy), C₃₋₁₀heterocyclyloxy (e.g., piperidinyloxy, cyclopentylpiperidinyloxy), 2, 3, or 4-halocycloalkyl (e.g., chlorophenyl), hydroxylC₁₋₈alkyl (e.g., hydroxylbutyl), aminoC₁₋₈alkylsulphonyl (e.g., aminomethylsulfonyl), C₁₋₈alkylsulphonyl (e.g., methyl sulfonyl), aminosulfonyl (e.g., sulfonamide), sulfonaminyl (e.g., sulfonamide), C₁₋₈alkylsulfonaminyl (e.g., methylsulfonamide), C₃₋₁₀cycloalkylacetamide (e.g., phenylacetamide), C₃₋₁₀cycloalkylsulfonaminyl (e.g., benzenesulfonamide, benzylsulfonamide, N-methylbenzenesulfonamide), heterocyclyl, anilinyl, C₁₋₈alkylanilinyl (e.g., methylanilinyl), imidazolyl (e.g., imidazol-1, 2, 3, or 4-yl), pyridyl (e.g., pyridin-1, 2, 3, or 4-yl), piperidinyl (e.g., piperidin-1, 2, 3, or 4-yl), piperidinylcarbonyl (e.g., piperidin-1, 2, 3, 4-ylcarbonyl), C₃₋₁₀cycloalkylpiperidinyl (e.g., phenylpiperidin-1, 2, 3, or 4-yl), pyrrolidyl (e.g., pyrrolidin-1, 2, or 3-yl), pyrazolyl (e.g., pyrazol-1, 2, 3, 4, or 5-yl), C₃₋₁₀cycloalkylpyrrolidinyl (e.g., 2, 3, or 4-phenylpyrrolidin-1, 2, or 3-yl), pyrimidinyl (e.g., pyrimidin-2,4, or 5, -yl), azaspiroheptanyl (optionally substituted with N, O, or S) (e.g., oxa-azaspiroheptanyl), or oxazolyl (e.g., oxa-2, 3, 4, or 5-yl).

Each of formula (I), R¹, R², R³, R⁴, R⁵, or R⁶ (or a ring formed from the aforementioned R groups) can be functionalized with, can comprise a linker group of, or can be substituted by, one or more groups selected from the group consisting of hydroxyl; C₁₋₁₀alkyl hydroxyl; aminyl; C₁₋₁₀carboxylic acid; C₁₋₁₀carbonyl, C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl; phenyl; heteroaryl containing from 1 to 4 N, O, or S atoms; unsubstituted phenyl ring; substituted phenyl ring; unsubstituted heterocyclyl; or substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; or heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; aryl comprising a phenyl; heteroaryl containing from 1 to 4 N, O, or S atoms; or a substituted bicyclic group (e.g., indole, azobicyclo, bridged bicyclic), or combinations thereof. Any of the above can be further functionalized or substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include-OH. The “hydroxyl” can be optionally substituted.

The terms “halogen” and “halo”, as used herein, unless otherwise indicated, include a chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, include a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “carbonyl”, as used herein, unless otherwise indicated, can include a functional group consisting of a carbon atom double-bonded to an oxygen atom (C═O). The “carbonyl” can be optionally substituted.

The term “alkenyl”, as used herein, unless otherwise indicated, can include alkyl moieties having at least one carbon-carbon double bond wherein alkyl is as defined above and including E and Z isomers of the alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O-cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated. The “alkoxyl” can be optionally substituted.

The term “cycloalkyl”, as used herein, unless otherwise indicated, can include an aromatic, a non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C₃₋₁₀ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include-lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S, and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with formula C₈H₇N. It has a bicyclic structure, consisting of a six-membered benzene ring fused to a five-membered nitrogen-containing pyrrole ring. The “indole” group can be optionally substituted.

The term “azabicyclo”, as used herein, indicates a bicyclic (e.g., fused or bridged bicyclic) structure in which a carbon atom has been replaced by a nitrogen atom. The “azabicyclo” group can be optionally substituted.

The term “bridged bicyclic”, as used herein, indicates a bicyclic structure in which the two rings share three or more atoms. The “bridged bicyclic” group can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such a compound. Examples of solvates include compounds of the invention in combination with, for example, water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. In instances where multiple charged atoms are part of the pharmaceutically acceptable salt, the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Autophagy Modulating Agent Activity

An autophagy modulating agent can be an agent having autophagy modulating activity. As an example, the autophagy modulating agent can be an active autophagy enhancing agent having enhanced autophagic degradation activity. Enhanced autophagic degradation (autophagy activity) of ATZ can be measured in a number of ways, but here, it was shown most definitively, by showing that the drug effect was blocked when the autophagy gene ATG14 was deleted in a mammalian cell line model of ATD. As an example, the measurement of decreased hepatic ATZ load together with increased LC3-II conversion and decreased p62 levels in the liver of the PiZ mouse model of ATD can be used as markers of increased autophagic activity.

As another example, an “active” autophagy enhancing agent can be an agent, when administered to a pre-clinical model (e.g., a mammalian cell line, C. elegans model of ATD using human ATZ, a PiZ mouse model) or a subject, can reduce the accumulation of misfolded, aggregated ATZ, reduce hepatic ATZ load, reduce p62 levels, increase LC3-II conversion, or reduce neuronal death when compared to a control or pre-clinical model or subject when administered a control or when there was no administration of the drug.

Measurements of autophagy-associated markers can be performed using an assay such as immunoblotting (e.g., Western Blot) or immunostaining (e.g., immunohistochemistry). An “active” autophagy enhancing agent can be an agent, when administered to striatal neurons from a Huntington's patient, reduces neuronal cell death compared to a control.

As such, an “active” autophagy enhancing agent can be an agent that, after administration to a cell or subject, results in reduced accumulation of misfolded, aggregated ATZ, reduced hepatic ATZ load, or reduced neuronal cell death compared to the administration of a control or no administration of an autophagy enhancing agent.

Activity can also be evaluated in comparison to GLB activity or other GLB analogs.

Methods for measuring autophagy activity can be as described in Yoshii and Mizushima 2017 Int J Mol Sci. 18 (9) 1865. Except as otherwise noted herein, therefore, the process of the present disclosure can be carried out in accordance with such processes.

### Formulation

The agents and compositions described herein can be formulated in any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating or preventing an autophagy-associated disease, disorder, or condition in a subject in need of administration of a therapeutically effective amount of an autophagy modulating agent, or combinations thereof, so as to modulate autophagy. The experiments using GLB analogs in the PiZ mouse model and in the striatal neurons, not only provide evidence for treatment, but also represent evidence of prevention in pre-clinical models. Modulation of autophagy can result in a reduction of proteotoxicity, liver damage, hepatic ATZ load, or liver fibrosis and improvement in neuronal survival.

Methods described herein are generally performed on a subject in need thereof. A subject in need of the therapeutic methods described herein can be a subject having, diagnosed with, suspected of having, or at risk for developing an autophagy-associated disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of an autophagy modulating agent is, for example, that amount that would cause the desired therapeutic effect in a subject while minimizing undesired side effects. In various embodiments, an effective amount of an autophagy modulating agent, described herein can substantially inhibit an autophagy-associated disease, slow the progress of an autophagy-associated disease, or limit the development of an autophagy-associated disease.

According to the methods described herein, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal administration.

When used in the treatments described herein, a therapeutically effective amount of an autophagy modulating agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to modulate autophagy.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an autophagy modulating agent can occur as a single event or over a time course of treatment. For example, an autophagy modulating agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for autophagy-associated disease.

An autophagy modulating agent can be administered simultaneously or sequentially with another agent, such as an antibiotic, an anti-inflammatory, or another agent. For example, an autophagy modulating agent can be administered simultaneously with another agent, such as an antibiotic or an anti-inflammatory. Simultaneous administration can occur through administration of separate compositions, each containing one or more of an autophagy modulating agent, an antibiotic, an anti-inflammatory, or another agent. Simultaneous administration can occur through administration of one composition containing two or more of an autophagy modulating agent, an antibiotic, an anti-inflammatory, or another agent. An autophagy modulating agent can be administered sequentially with an antibiotic, an anti-inflammatory, or another agent. For example, an autophagy modulating agent can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22 (3): 659-661 , 2008, which is incorporated herein by reference):

HED(mg/kg)=Animal dose(mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, an autophagy modulating agent may be administered to a human patient in an amount from about 1 mg to about 1000 mg, or about 1 mg to about 900 mg, or about 1 mg to about 800 mg, or about 1 mg to about 700 mg, or about 1 mg to about 600 mg, or about 1 mg to about 500 mg, or about 1 mg to about 400 mg, or about 1 mg to about 300 mg, or about 1 mg to about 200 mg, or about 1 mg to about 100 mg, or about 1 mg to about 75 mg, or about 1 mg to about 50 mg, or about 1 mg to about 25 mg, or about 1 mg to about 20 mg, or about 1 mg to about 15 mg, or about 1 mg to about 10 mg, or about 1 mg to about 5 mg, or about 1 mg to about 3 mg. In some embodiments, an autophagy modulating agent such as a compound described herein may be administered in a range of about 1 mg to about 100 mg, or about 50 mg to about 250 mg, or about 100 mg to about 500 mg, or about 250 mg to about 750 mg, or about 500 mg to about 1000 mg, or about 1000 mg.

The effective amount in a human patient may be in the range of 1 mg/day to 1000 mg/day. In some embodiments, the effective amount may be less than or about 0.75 mg/day, 1.5 mg/day, 2.5 mg/day. 5 mg/day, 10 mg/day, 20 mg/day, 25 mg/day, 50 mg/day, 75 mg/day, 100 mg/day, 200 mg/day, 250 mg/day, 300 mg/day, 400 mg/day, 500 mg/day, 600 mg/day, 700 mg/day, 800 mg/day, 900 mg/day, or 1000 mg/day.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for drug screening or genetic screening for autophagy modulators.

A C. elegans model of ATD using human ATZ or and a PiZ mouse model can be used. A compound or genetic information can be introduced to the model and the value of ATZ accumulation or reduction can be measured.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., a molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., a molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Compositions of Autophagy Enhancing Agents

Alpha1-antitrypsin deficiency (ATD) is an inherited disorder that can result in liver disease. It was discovered that glibenclamide (GLB), an FDA approved drug for type 2 diabetes enhances the autophagic degradation of misfolded ATZ and therein is a potential therapeutic for ATD. A series of GLB analogs, referred to herein as Ex 1-116, (shown below) have been created. These GLB analogs reduced hepatic ATZ load and fibrosis in the PiZ mouse model of ATD without affecting insulin secretion and decrease cellular ATZ load in the C. elegans model of ATD in a dose-dependent fashion. Additionally, GLB analogs improved the survival of human striatal neurons derived from patients with Huntington's disease.

Taken together these results are consistent with an effect of the analog on two different types of misfolded proteins and two different types of misfolded diseases, as would be predicted for an autophagy enhancer drug. The data also suggests that this analog and derivatives could be a therapeutic for misfolded protein diseases and age-dependent degenerative diseases. A new series of analogs described here, have increased potency and stability.

Examples display biological activity ranging from about 0.001 μM to about 10.00 μM in the HTO/Z and/or ICW assays.

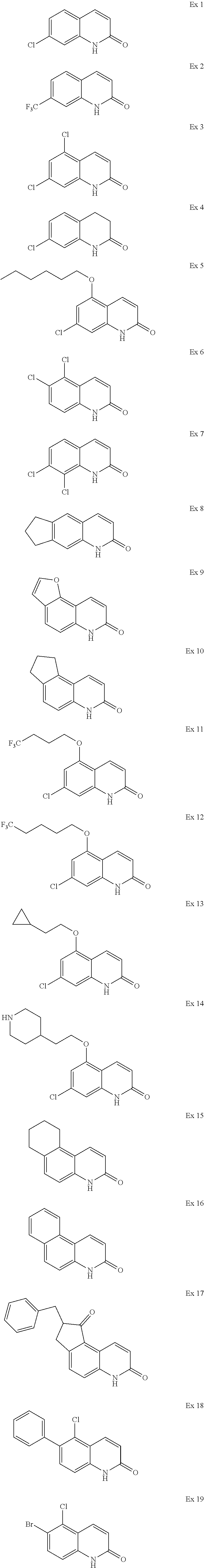

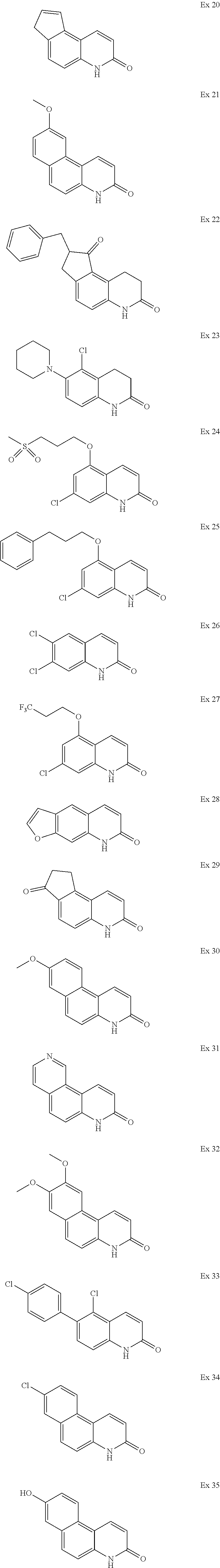

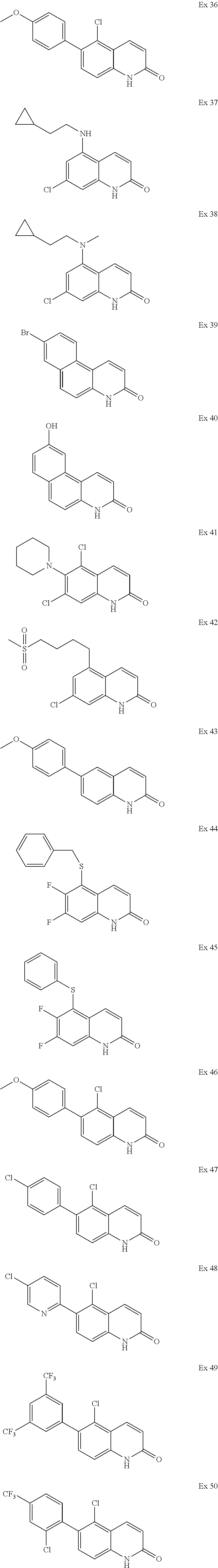

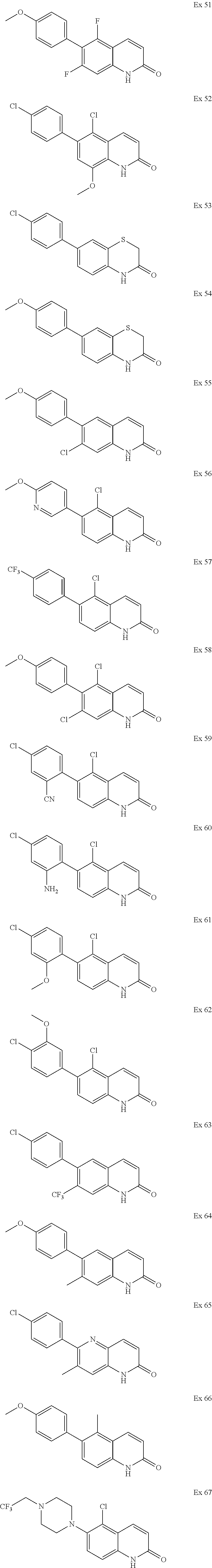

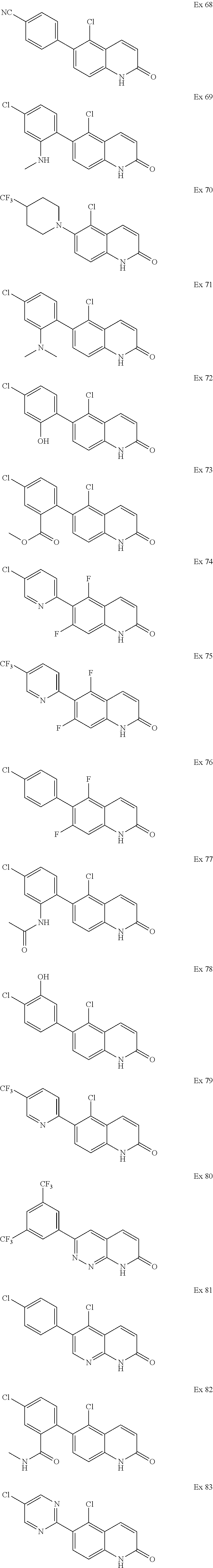

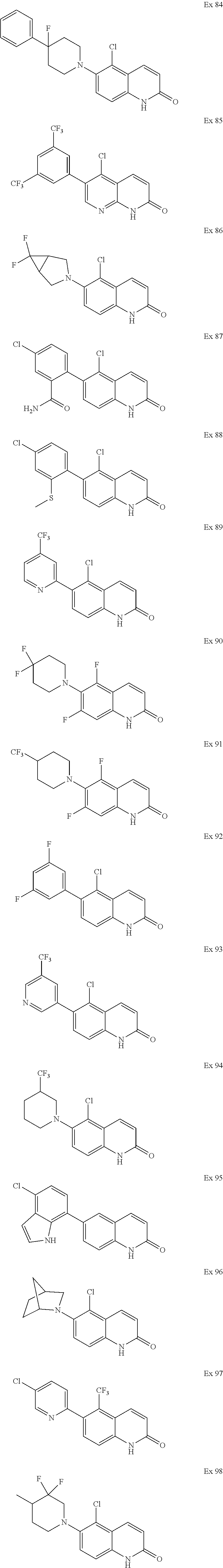

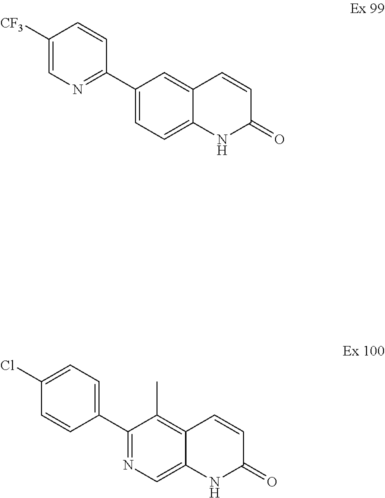 [SMILES: O=c1ccc2cc(-c3ccc(C(F)(F)F)cn3)ccc2[nH]1 ; Cc1c(-c2ccc(Cl)cc2)ncc2[nH]c(=O)ccc12]

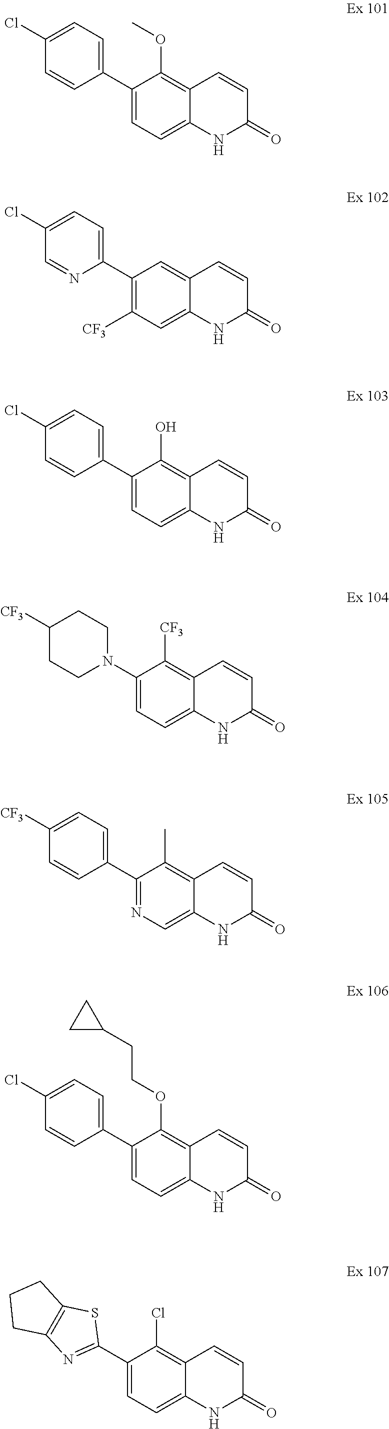

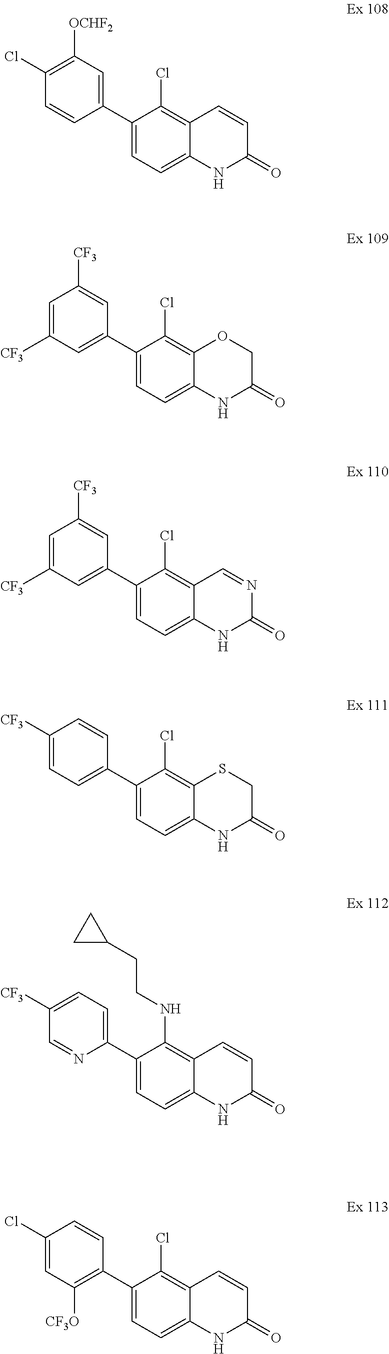

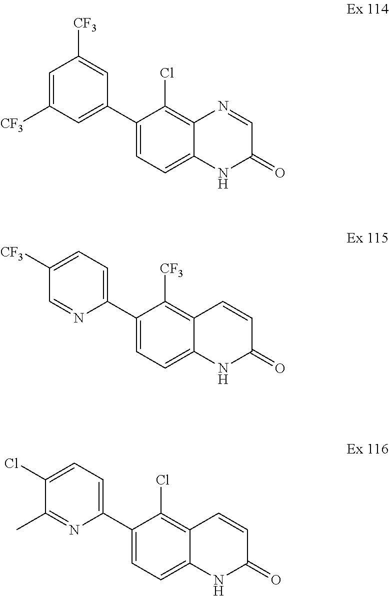 [SMILES: O=c1cnc2c(Cl)c(-c3cc(C(F)(F)F)cc(C(F)(F)F)c3)ccc2[nH]1 ; O=c1ccc2c(C(F)(F)F)c(-c3ccc(C(F)(F)F)cn3)ccc2[nH]1 ; Cc1nc(-c2ccc3[nH]c(=O)ccc3c2Cl)ccc1Cl]

The 5,6-disubstituted series are promising candidates for optimization (e.g., optimizing this series for ADME, PK, and physiochemical properties). In exemplary embodiments, an autophagy modulating agent is a 5,6-disubstituted quinolone, such as selected from, but not limited, to Ex 6, Ex 18, Ex 19, Ex 33, Ex 36, Ex 47, Ex 48, Ex 49, Ex 56, or Ex 97.

### Example 2: GLB Analog Effects and Ex 3 Increased the Resilience of Hd-MSNS Against Neuronal Death by Promoting Autophagy

This example describes GLB analogs and their effects on AZT levels and autophagy.

Direct neuronal conversion of fibroblasts from Huntington's disease (HD) patients to striatal medium spiny neurons (MSNs) has been shown to recapitulate neurodegenerative pathology of HD. Herein is shown that treating with Ex 3, a glibenclamide (GLB) analog, increased the resilience of HD-MSNs against neuronal death by promoting autophagy towards the pre-HD-MSN state, demonstrating that the autophagic decline during aging in HD underlies MSN degeneration and pointing to potential approaches for enhancing autophagy and resilience of MSNs against degeneration in HD.

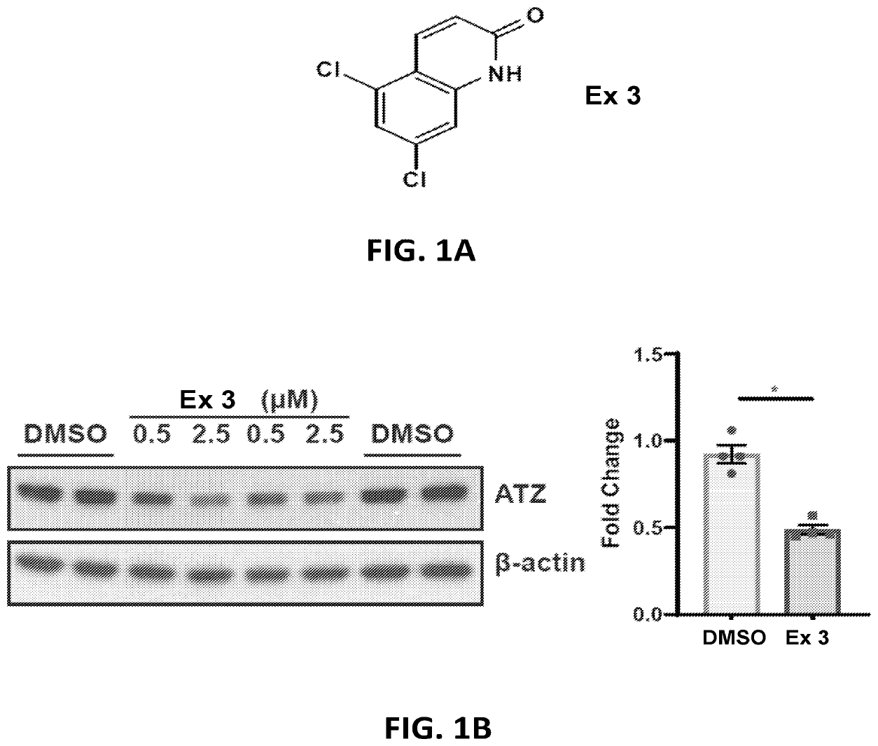

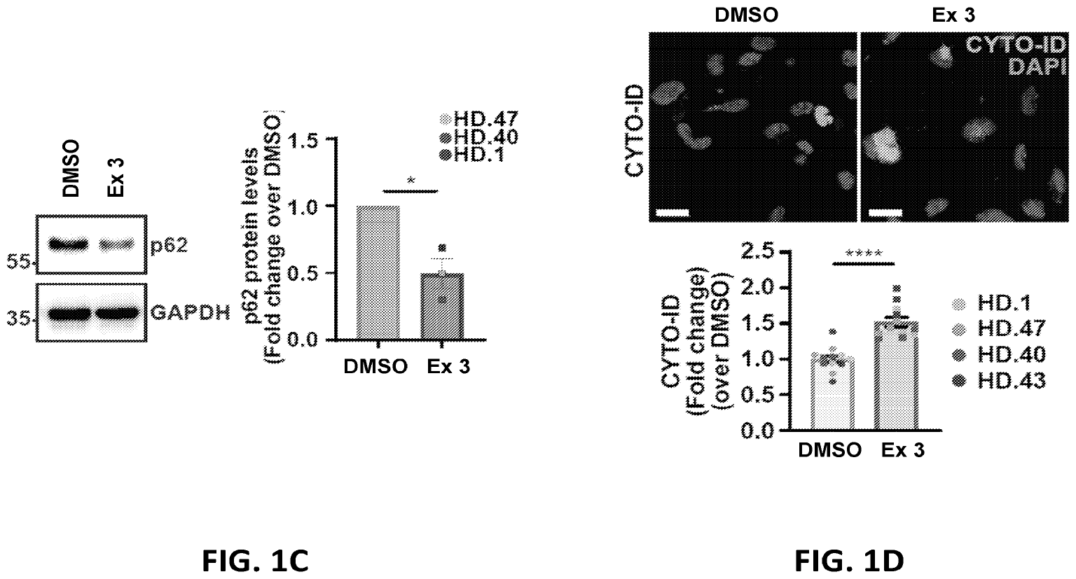

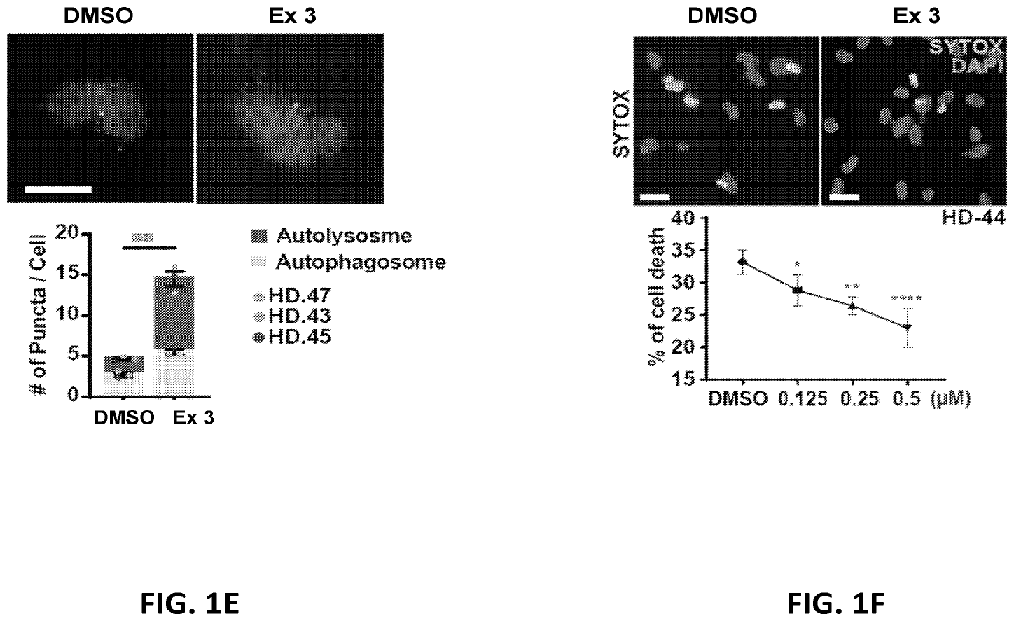

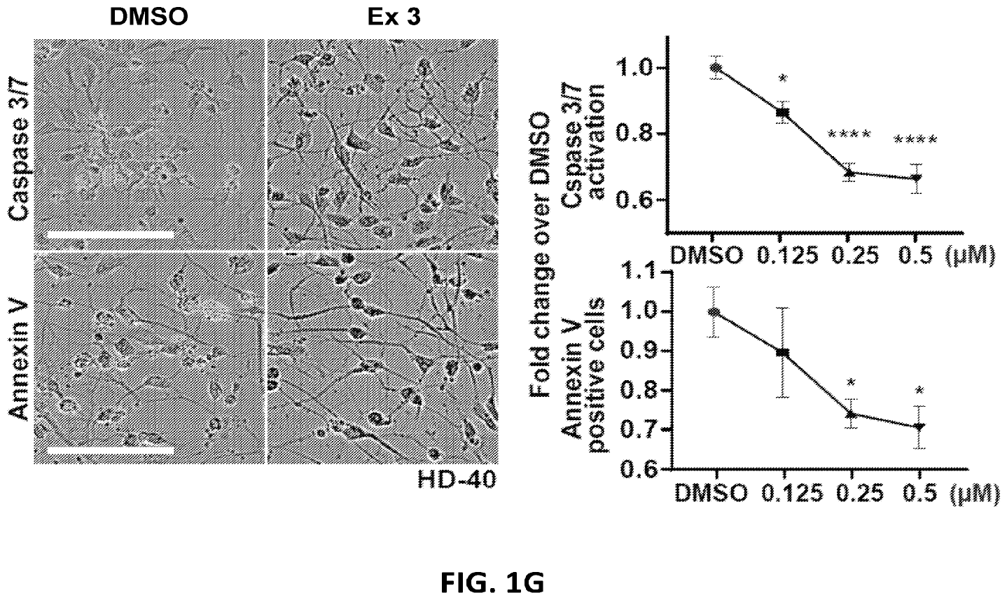

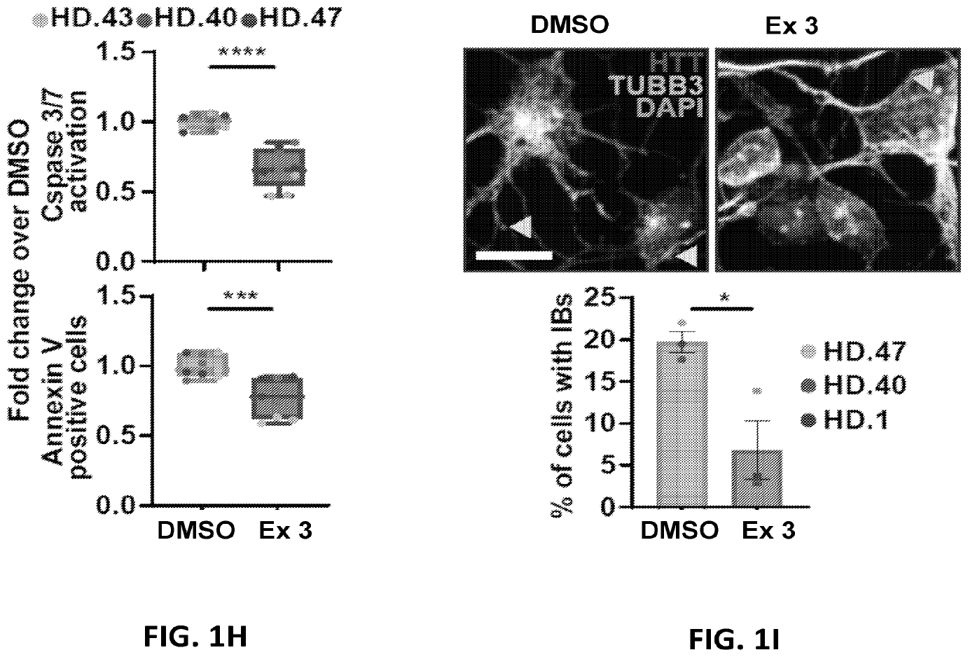

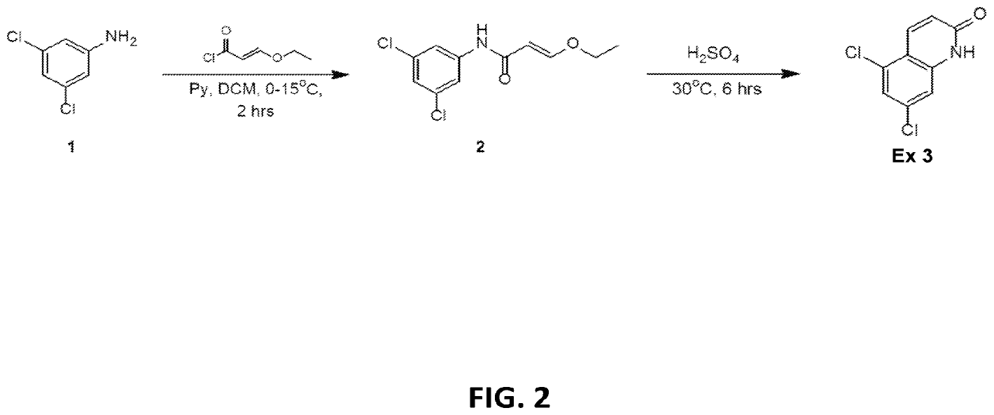

It was tested if overriding the autophagy deficiency in MSNs derived from symptomatic patients (HD-MSNs) would shift the degeneration state toward pre-HD-MSNs. For this, a new analog of glibenclamide (GLB), a sulfonylurea drug that has been used broadly in clinics as an oral hypoglycemic agent, was developed. An initial GLB analog, “G2”, was previously shown to promote autophagic degradation of misfolded α1-antitrypsin Z variant (ATZ) in mammalian cell models of α1-antitrypsin deficiency (ATD) disorder. The new GLB analog tested herein (Ex 3), designed to increase the potency of the compound (see e.g., FIG. 1A and FIG. 2), decreased the steady-state levels of ATZ in a HTO/Z cell line model of ATD (see e.g., FIG. 1B). Treating HD-MSNs with Ex 3 decreased p62/SQSTM1 protein levels (see e.g., FIG. 1C) and increased the number of autophagic vacuoles as measured by CYTO-ID signals (see e.g., FIG. 1D). Also, Ex 3 increased the average number of autophagosomes and autolysosomes in HD-MSNs expressing the RFP-GFP-LC3 reporter (see e.g., FIG. 1E), verifying the autophagy-enhancing activity of Ex 3 in HD-MSNs. Cells were treated with Ex 3 at PID14, a time point when cells undergoing miRNA-mediated conversion adopt neuronal identity, and further treated every four days for 16 days. Importantly, Ex 3 resulted in a significant reduction in HD-MSN death in a dose-dependent manner, as measured by the SYTOX assay (see e.g., FIG. 1F) and in activated Caspase 3/7 and Annexin V signals in HD-MSNs at PID30 (see e.g., FIG. 1G). This result was confirmed in three other independent lines of HD-MSNs (see e.g., FIG. 1H). Ex 3 also reduced the number of mHTT inclusion bodies in HD-MSNs compared to DMSO treatment (see e.g., FIG. 1I). Together, this data demonstrates that enhancing autophagy in HD-MSNs alleviates the degenerative state. These findings are interesting because in mouse embryonic fibroblasts harboring the repeat number in the range of 100, mutant HTT was shown to fail to be loaded into autophagosomes. The results herein, however, challenge this notion such that in neurons derived from adult-onset patients, enhancing autophagy increases HD-MSNs' ability to clear mHTT aggregation and their resilience against neurodegeneration.

### Synthetic Route for the Preparation of Ex 3 (FIG. 2).

Preparation of intermediate 2. To a solution of 3,5-dichloroaniline (2 g, 12.34 mmol, 1 eq.) in DCM (30 mL) was added pyridine (3.42 g, 43.21 mmol, 3.5 eq.) and 3-ethoxyprop-2-enoyl chloride (1.99 g, 14.81 mmol, 1.2 eq.) at 0° C. The mixture was stirred at 15° C. for 2 hr. The reaction mixture was quenched by aqueous HCl (1M, 10 mL) and extracted with DCM (30 mL×3). The combined organic layers were washed with aq. sat. NaHCO₃ (15 mL), dried over Na₂SO₄, filtered and concentrated to give (E)-N-(3,5-dichlorophenyl)-3-ethoxy-prop-2-enamide 2 (3.1 g, crude) as a yellow solid. ESI [M+H]=260.0

Preparation of Ex 3. A mixture of 2 (1.5 g, 5.77 mmol, 1 eq.) in sulfuric acid (15 mL, 98% purity) was stirred at 30° C. for 6 hr. The reaction mixture was poured into ice water (30 mL) and filtered. The filter cake was triturated with MeOH (8 mL×3) and the filter cake was dried to give 5,7-dichloro-1H-quinolin-2-one Ex 3 (1.1 g, 4.97 mmol, 86.1% yield, 96.6% purity) as a brown solid. ¹H NMR (400 MHZ, DMSO-d6) δ=12.72-11.45 (m, 1H), 8.05 (br d, J=9.8 Hz, 1H), 7.48 (s, 1H), 7.33 (s, 1H), 6.71-6.59 (m, 1H). ESI [M+H]=214.0/216.0

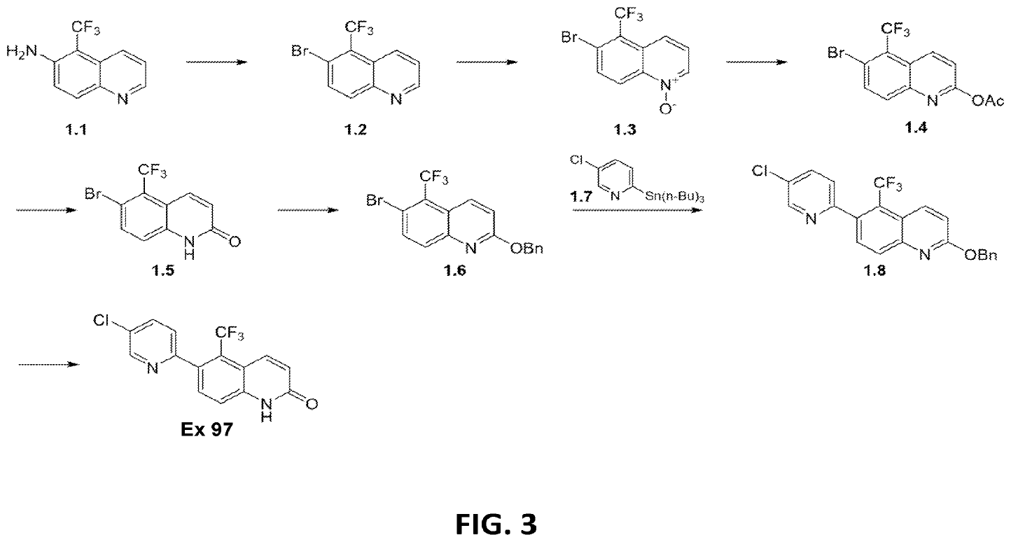

### Synthetic Route for the Preparation of Ex 97 (FIG. 3).

To a solution of 5-(trifluoromethyl) quinolin-6-amine 1.1 (16.00 g, 75.42 mmol, 1.00 eq) in acetonitrile (1500.0 mL) was added CuBr (14.06 g, 98.0 mmol, 2.98 mL, 1.30 eq), [(1S,4R)-7,7-dimethyl-2-oxo-norbornan-1-yl]methanesulfonic acid (52.56 g. 226.24 mmol, 3.00 eq), tetrabutylammonium bromide (145.86 g, 452.46 mmol, 6.00 eq), NaNO₂ (15.6 g, 226.24 mmol, 3 eq) at 0° C. The reaction mixture was stirred at 25° C. for 12 h. The reaction mixture was poured into water (500 mL) and extracted with ethyl acetate (1 L×3). The combined organic phase was washed with brine (500 mL×3), dried with anhydrous Na₂SO₄, filtered, and concentrated under reduced pressure. The residue was purified by column chromatography. Quinoline 1.2 (19.4 g, 70.28 mmol, 93.19% yield) was obtained as a green oil. MS: m/z 275.96 (M+H)+. 1H NMR (400 MHZ): DMSO δ 9.07 (dd, J=1.2, 4.1 Hz, 1H), 8.65-8.51 (m, 1H), 8.26-8.03 (m, 2H), 7.76 (dd, J=4.2, 8.9 Hz, 1H).

To a solution of quinoline 1.2 (19.40 g, 70.27 mmol, 1.00 eq) in dichloromethane (500 mL) was added meta-chloroperbenzoic acid (21.40 g, 105.42 mmol, 85% purity, 1.50 eq) at 0° C. The reaction mixture was stirred at 25° C. for 12 h. The reaction mixture was quenched by saturated aqueous Na₂SO₃ (1 L) at 0° C. and extracted with dichloromethane (500 mL×3). The combined organic phase were washed with saturated aqueous Na₂CO₃ (500 mL×3), dried with anhydrous Na₂SO₄, filtered, and concentrated under reduced pressure. The residue was used in the next step without any purification. Oxynitride 1.3 (19.80 g, crude) was obtained as yellow solid. MS: m/z 291.95 (M+H)+. 1H NMR (400 MHz): DMSO δ 8.82-8.64 (m, 2H), 8.17 (d, J=9.4 Hz, 1H), 8.06 (br dd, J=1.2, 9.2 Hz, 1H), 7.67 (dd, J=6.1, 9.1 Hz, 1H).

To a solution of oxynitride 1.3 (19.80 g, 67.79 mmol, 1 eq) in acetic anhydride (200 mL) was stirred at 140° C. for 12 h. The reaction mixture was concentrated under reduced pressure to give a residue. The residue was used in the next step without any purification. 6-bromo-5-(trifluoromethyl) quinolin-2-yl acetate 1.4 (22 g, crude) was obtained as brown oil. MS: m/z 333.96 (M+H)+

To a solution of 6-bromo-5-(trifluoromethyl) quinolin-2-yl acetate 1.4 (22 g, 65.85 mmol, 1 eq) in MeOH (400 mL) was added K₂CO₃ (18.20 g, 131.7 mmol, 2 eq) at 0° C. The mixture was stirred at 25° C. for 12 h. The reaction mixture concentrated under reduced pressure to give a residue. The residue was washed with water (250 mL), the mixture was filtered to obtain filter cake. The filter cake was washed with ACN (80 mL), and the filter cake concentrated under reduced pressure to give a residue. 6-bromo-5-(trifluoromethyl) quinolin-2 (1H)-one 1.5 (16 g, 54.78 mmol, 83.2% yield) was obtained as a pink solid. MS: m/z 291.95 (M+H)+. 1H NMR (400 MHZ): DMSO δ 8.03 (dd, J=1.6, 10.1 Hz, 1H), 7.86 (d, J=8.9 Hz, 1H), 7.51 (d, J=8.9 Hz, 1H), 6.71 (d, J=10.1 Hz, 1H).

To a solution of 6-bromo-5-(trifluoromethyl) quinolin-2 (1H)-one 1.5 (16.00 g, 54.78 mmol) and benzylbromide (11.24 g, 65.74 mmol, 7.80 mL, 1.20 eq) in Tol. (500 mL) was added Ag₂CO₃ (30.20 g, 109.56 mmol, 4.97 mL, 2.00 eq). The reaction mixture was stirred at 70° C. for 12 h under N₂. The reaction mixture was filtered and concentrated under reduced pressure to give a residue. The residue was purified by column chromatography. 2-(benzyloxy)-6-bromo-5-(trifluoromethyl) quinoline 1.6 (15.4 g, 40.3 mmol, 74% yield) was obtained as a white solid. MS: m/z 382.00 (M+H)+. 1H NMR (400 MHZ): DMSO δ 8.48 (dd, J=1.6, 9.5 Hz, 1H), 8.09-8.02 (m, 1H), 7.99-7.93 (m, 1H), 7.58-7.51 (m, 2H), 7.45-7.27 (m, 4H), 5.54 (s, 2H).

To a solution of 2-(benzyloxy)-6-bromo-5-(trifluoromethyl) quinoline 1.6 (10 g, 26.16 mmol, 1 eq) and tributyl-(5-chloro-2-pyridyl) stannane (13.7 g, 34.02 mmol, 1.3 eq) in dioxane (300 mL) was added Pd(dppf)Cl₂ (967.3 mg, 1.31 mmol, 0.05 eq). The reaction mixture was stirred at 100° C. for 12 h under N₂. The reaction mixture was filtered and concentrated under reduced pressure to give a residue. The residue was purified by column chromatography. 2-(benzyloxy)-6-(5-chloropyridin-2-yl)-5-(trifluoromethyl) quinoline 1.7 (8.4 g, 20.25 mmol, 77.4% yield) was obtained as a white solid. MS: m/z 415.07 (M+H)+. 1H NMR (400 MHZ): DMSO δ 8.85-8.66 (m, 1H), 8.49 (br dd, J=2.0, 9.4 Hz, 1H), 8.25-7.99 (m, 2H), 7.80-7.52 (m, 4H), 7.51-7.19 (m, 4H), 5.59 (s, 2H).

To a solution of 2-(benzyloxy)-6-(5-chloropyridin-2-yl)-5-(trifluoromethyl) quinoline 1.7 (8.4 g, 20.25 mmol, 1 eq) in TFA (100 mL) was stirred at 60° C. for 0.5 h. The reaction mixture was concentrated under reduced pressure to give a residue. The residue was washed with saturated aqueous Na₂CO₃ (200 mL), the mixture was filtered to obtain filter cake. The filter cake was washed with ACN (50 mL), and the filter cake concentrated under reduced pressure to give a residue. 6-(5-chloropyridin-2-yl)-5-(trifluoromethyl) quinolin-2 (1H)-one Ex 97 (4.76 g, 14.61 mmol, 81.9% yield, 99.64% purity) was obtained as a white solid. MS: m/z 325.03 (M+H)+. 1H NMR (400 MHz): DMSO δ 12.29 (s, 1H), 8.71 (d, J=2.4 Hz, 1H), 8.14-8.02 (m, 2H), 7.68-7.63 (m, 1H), 7.61-7.54 (m, 2H), 6.79 (dd, J=1.5, 10.0 Hz, 1H).

As described herein, continued lead optimization afforded potent quinolones. The new series of quinolones described herein displayed durable structure-activity relationship (SAR) and are a rich source of active compounds. Quinolone Ex 3 is up to 50× more potent than the initial “G2” analog of GLB. The quinolone compounds also exhibit liver microsomal stability (se e.g., TABLE 1).

TABLE 1
Liver microsomal stability

| | |
|---|---|
| Cell | LM stability |

| | activity | Mouse | Human | |
|---|---|---|---|---|
| Compound | (HTO/Z) | t ½ (min) | t ½ (min) | MW |
| Ex 3 | ++ | 4.2 | ND | 214.05 |
| Ex 97 | +++ | >145 | >145 | 324.69 |

+ = about 1.0-10.0 μM
++ = about 0.10-1.0 μM
+++ = about 0.01-0.10 μM
ND = not determined

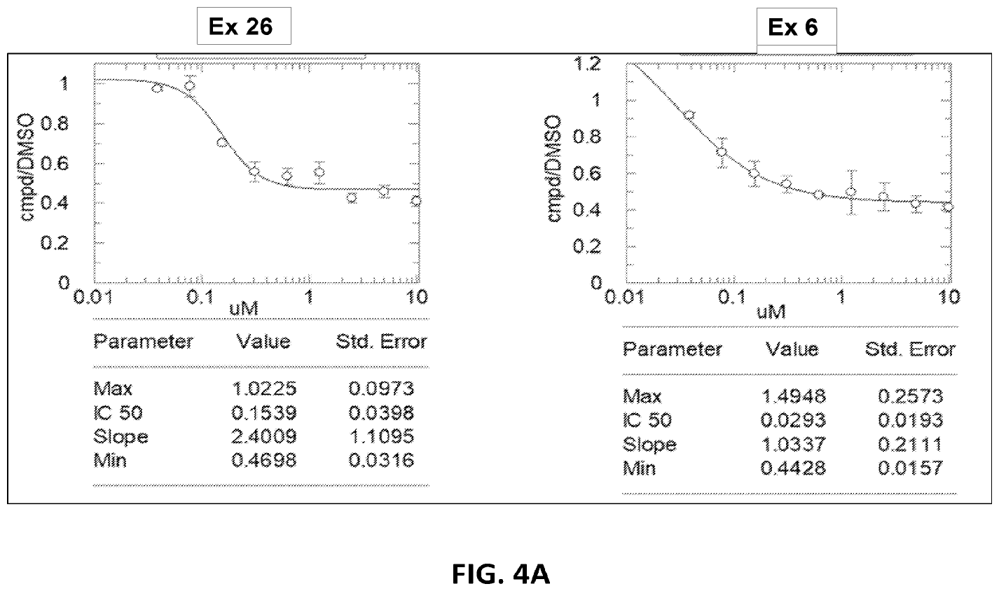

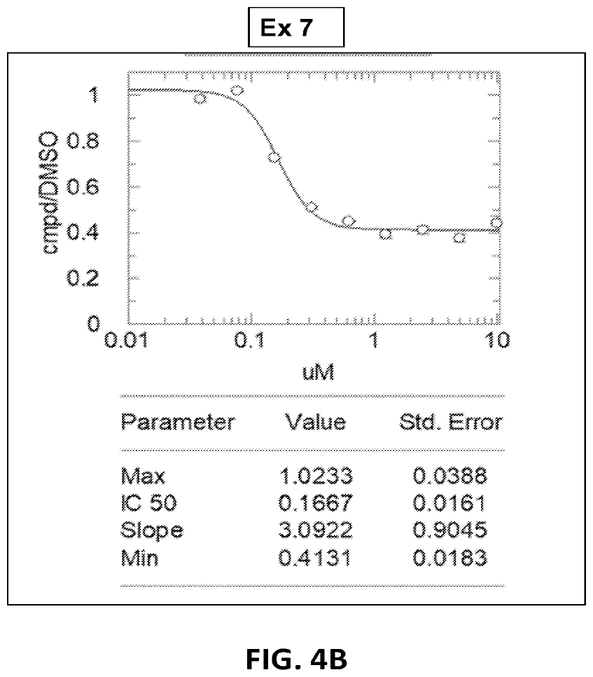

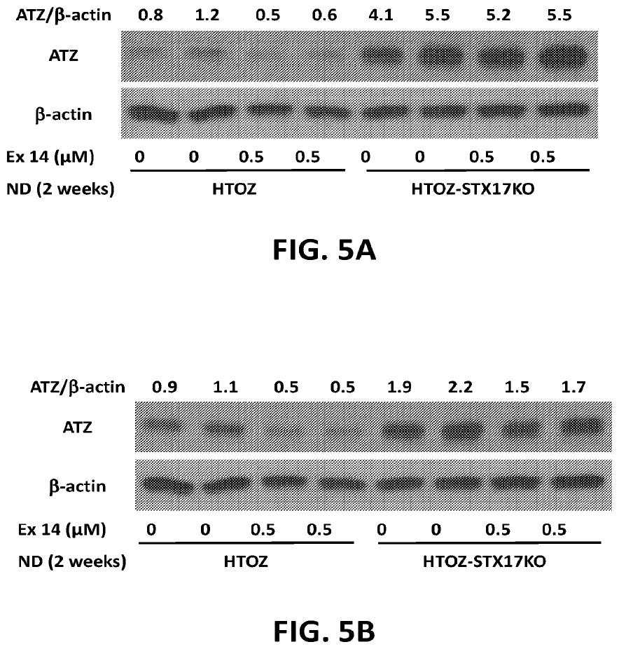

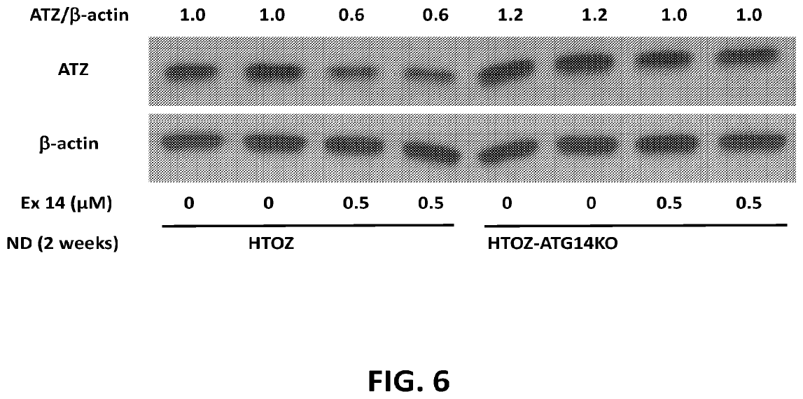

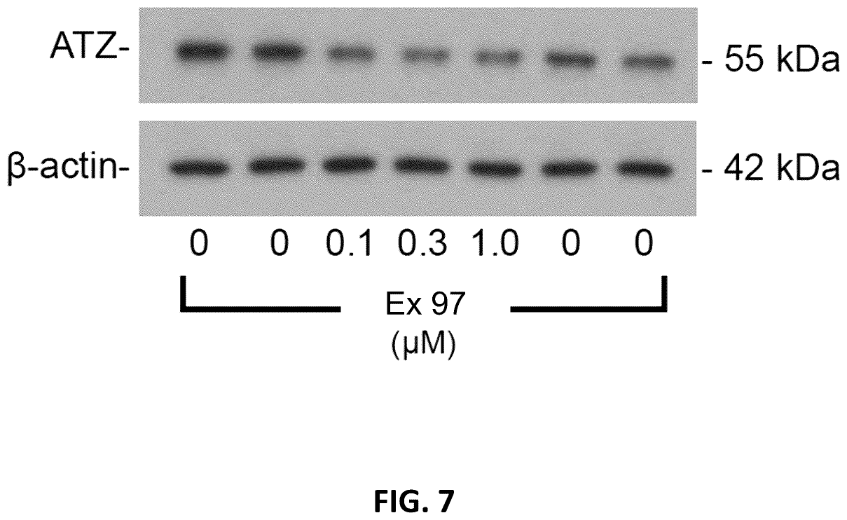

An In-Cell-Western assay with quinolone compounds Ex 26, Ex 6, and Ex 7, showed that these compounds are capable of inhibiting poly ATZ in HTOZ cells (see e.g., FIG. 4A and FIG. 4B). It was also found that GLB analogs (e.g., Ex 14) affected ATZ levels in a HTO/Z cell lines engineered to disrupt macro-autophagy (STX17KO) (see e.g., FIG. 5A-FIG. 5B) or (ATG14KO) (see e.g, FIG. 6).

What is claimed is:

1. A composition comprising an autophagy modulating agent selected from formula (I): 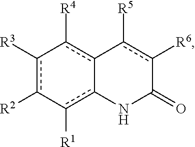
wherein R¹, R², R³, R⁴, R⁵, or R⁶ is hydrogen (H), halo (e.g., Cl, F), C₁₋₈alkyl (e.g., methyl, ethyl, butyl, propyl, isopropyl, isopentyl), halogen-substituted C₁₋₈alkyl (e.g., trifluoromethyl), piperidinyl (e.g., piperidin-1, 2, 3, or 4-yl), C₃₋₁₀cycloalkyl (e.g., phenyl), halogen-substituted C₃₋₁₀cycloalkyl (e.g., chlorophenyl), pyrimidinyl (e.g., pyrimidin-2,4, or 5, -yl), C₁₋₈alkoxy (e.g., methoxy, ethoxy, propoxy, butoxy, hexoxy), halogen substituted C₁₋₈alkyl (e.g., trifluoromethyl, trifluorobutoxy, trifluoropentoxy), C₁₋₆alkyl-C₃₋₁₀cycloalkoxy (e.g., cyclopropylethoxy), C₃₋₁₀cycloalkyl-C₁₋₆alkyl-amino (e.g., cyclopropylethylamino, cyclopropylethyl(methyl)amino), C₃₋₁₀heterocyclyloxy (e.g., piperidinyloxy, cyclopentylpiperidinyloxy), C₁₋₈alkylsulphonyl (e.g., methyl sulfonyl), C₁₋₈alkylsulphonylalkoxy (e.g., methyl sulfonylpropoxy), C₁₋₁₀cycloalkoxy (e.g., phenyl propoxy), alkoxyC₁₋₁₀cycloalkyl (e.g., methoxy phenyl), substituted phenyl; and/or
R¹ or R², R² or R³, or R³ or R⁴ optionally form a ring (e.g., heterocyclyl, cycloalkyl, furanyl, pyridinyl, phenyl, etc.); a 5 or 6 membered ring (e.g., pyridinyl, furanyl, cycloalkyl, heterocyclyl); a substituted 5- or 6-membered ring (e.g., an alkyl-, alkyl-phenyl-, hydroxyl-, halo-, chloro-, bromo-, methoxy-, di-methoxy-, oxy-, or benzyl-substituted 5 or 6 membered ring); or a substituted bicyclic group (e.g., indole, azobicyclo, bridged bicyclic).

2. The composition of claim 1, wherein the autophagy modulating agent is selected from:
(i) a 5,6-disubstituted quinolone;

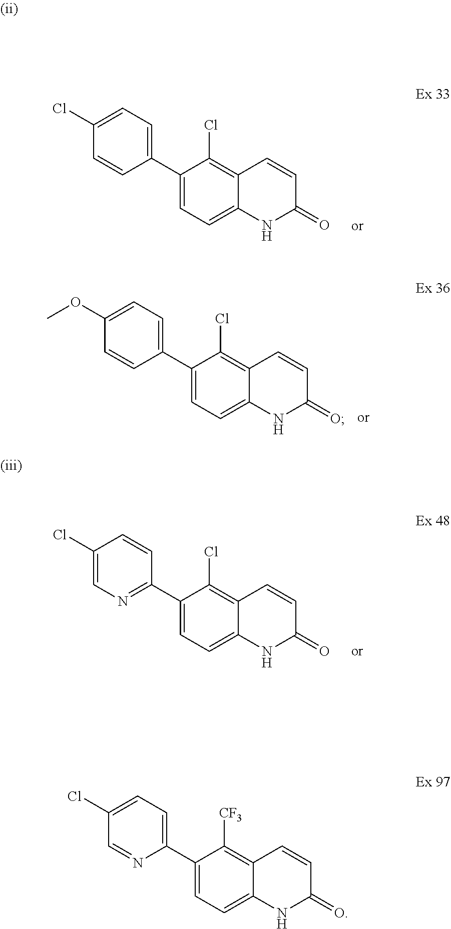 [SMILES: O=c1ccc2c(Cl)c(-c3ccc(Cl)cc3)ccc2[nH]1 ; COc1ccc(-c2ccc3[nH]c(=O)ccc3c2Cl)cc1 ; "or" ; O=c1ccc2c(Cl)c(-c3ccc(Cl)cn3)ccc2[nH]1 ; O=c1ccc2c(C(F)(F)F)c(-c3ccc(Cl)cn3)ccc2[nH]1]

3. The composition of claim 2, wherein the autophagy modulating agent is a 5,6-disubstituted quinolone.

4. The composition of claim 3, wherein the autophagy modulating agent is

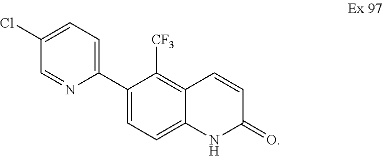 [SMILES: O=c1ccc2c(C(F)(F)F)c(-c3ccc(Cl)cn3)ccc2[nH]1]

5. The composition of claim 1, wherein the autophagy modulating agent is selected from the group consisting of: 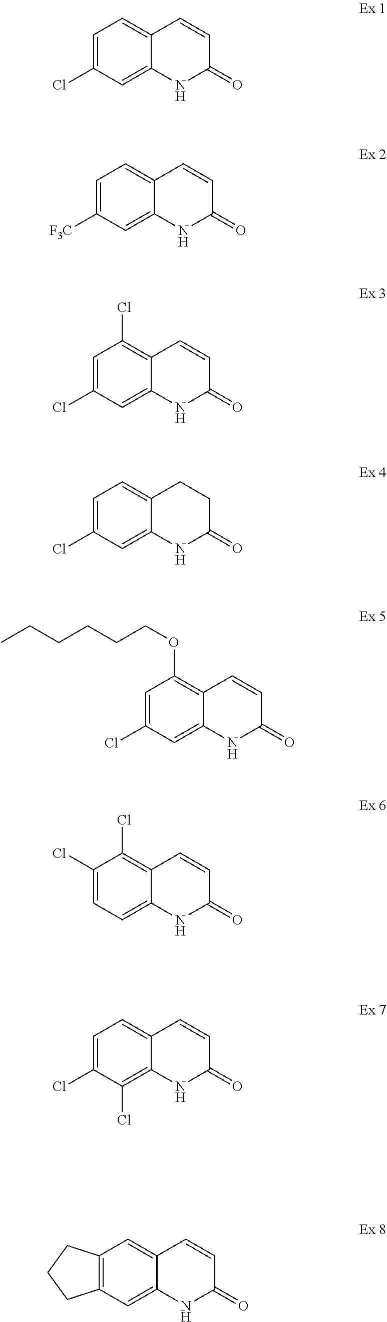 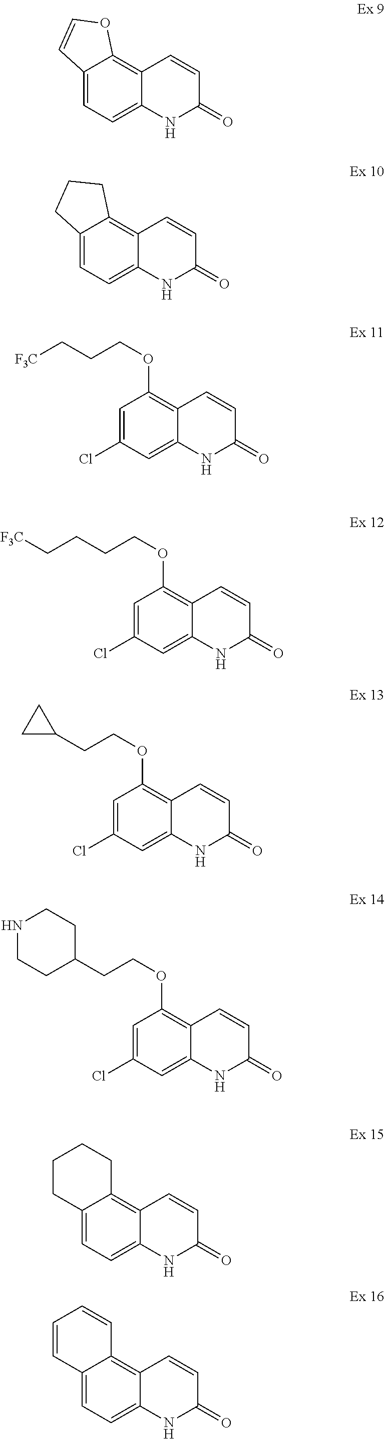 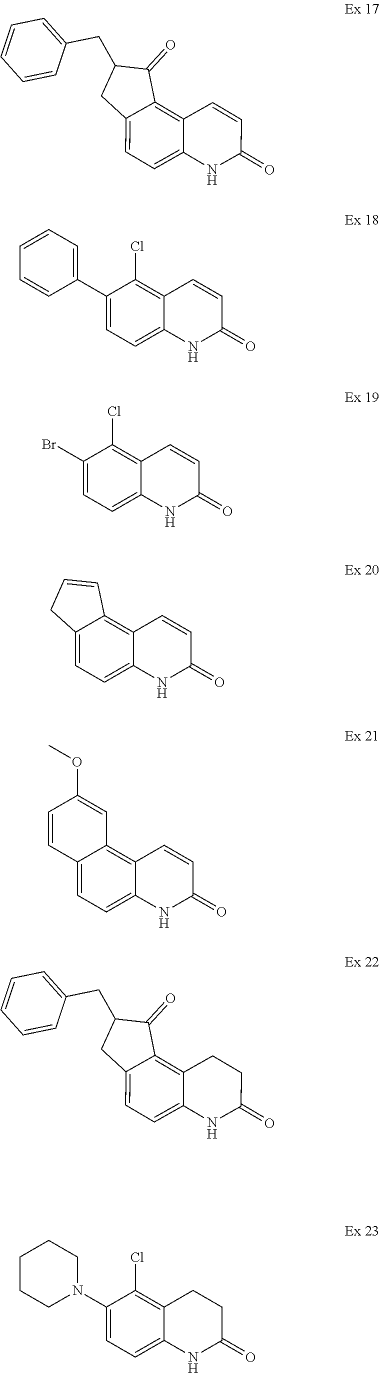 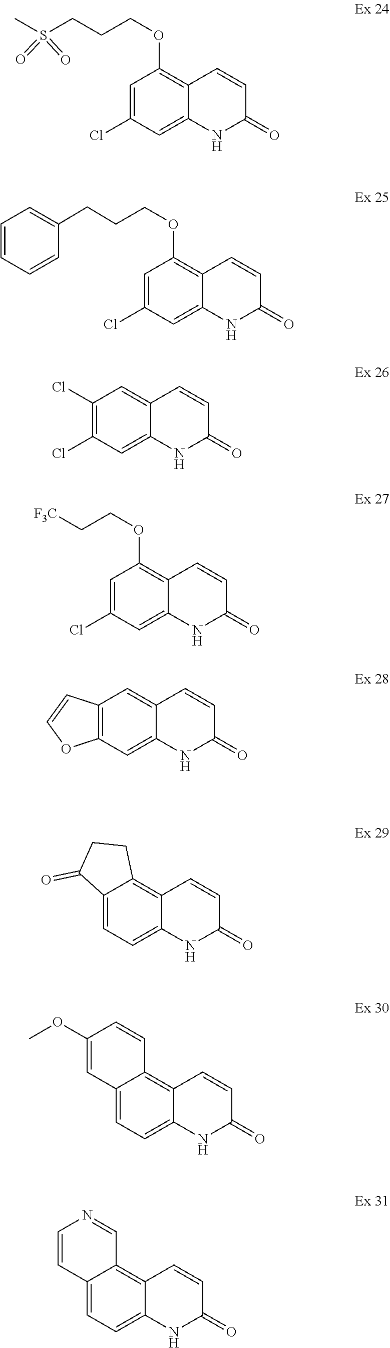 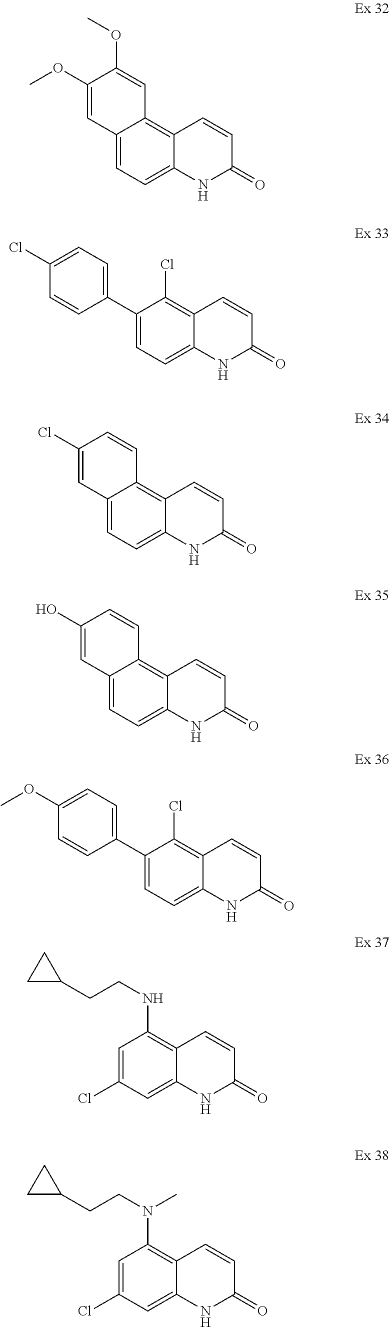 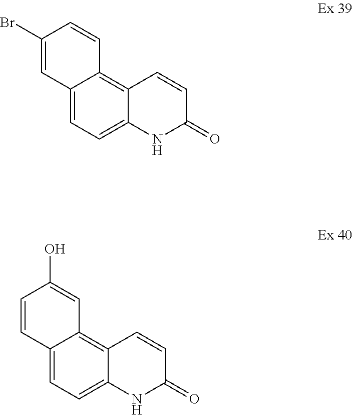 [SMILES: O=c1ccc2c(ccc3cc(Br)ccc32)[nH]1 ; O=c1ccc2c(ccc3ccc(O)cc32)[nH]1]
or a pharmaceutically acceptable salt thereof, including all tautomers and stereoisomers, and optionally substituted analogs thereof.

6. The composition of claim 1, wherein the autophagy modulating agent is selected from the group consisting of: 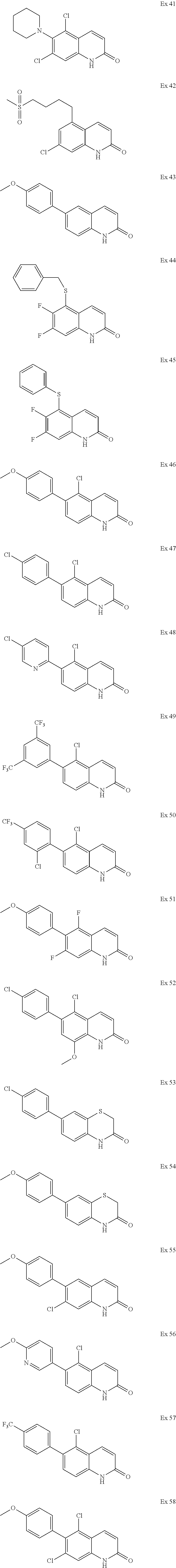 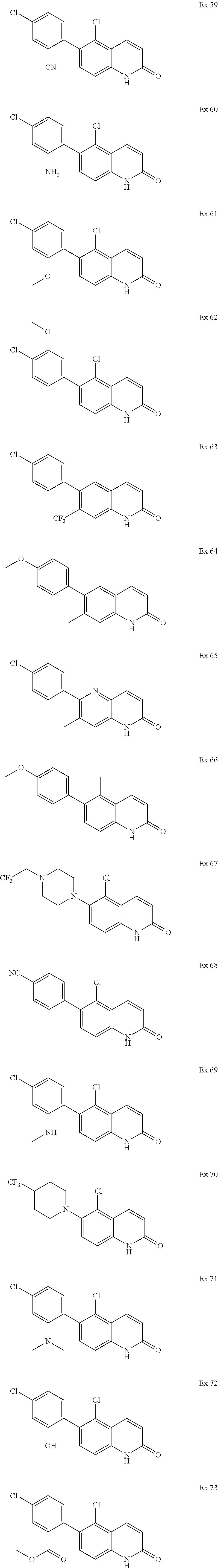 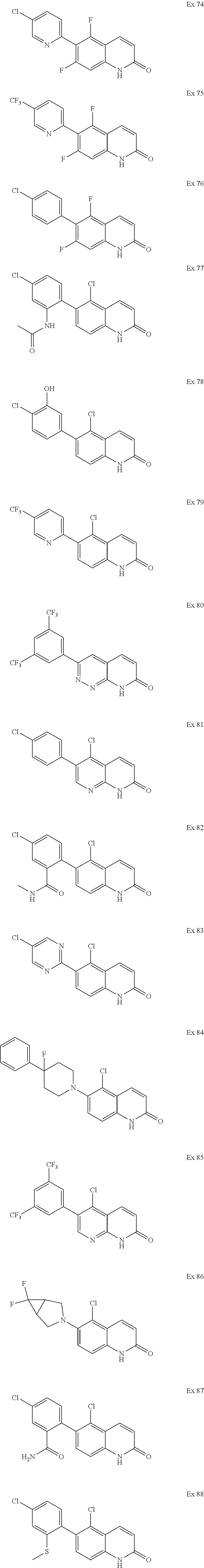 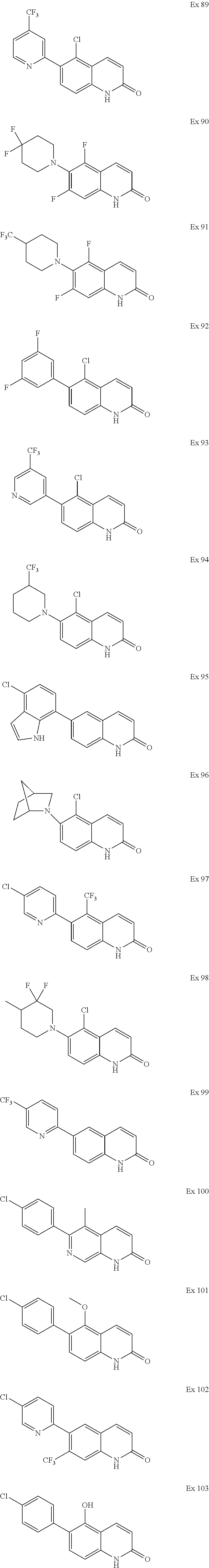 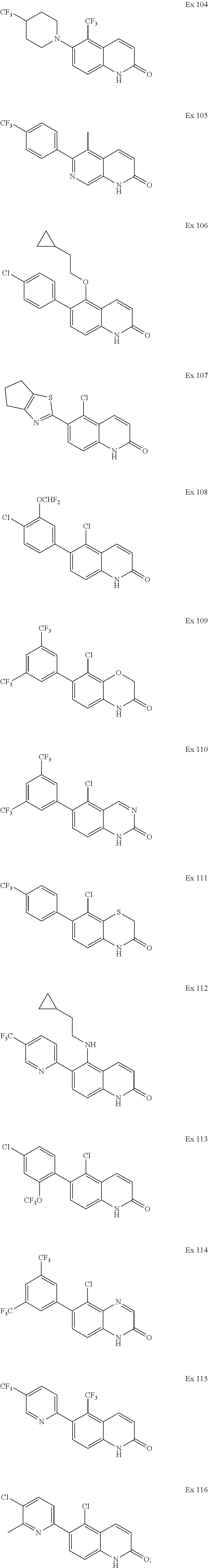
or a pharmaceutically acceptable salt thereof, including all tautomers and stereoisomers, and optionally substituted analogs thereof.

7. The composition of claim 1, wherein R¹ is hydrogen.

8. The composition of claim 1, wherein the autophagy modulating agent has autophagy modulating activity.

9. The composition of claim 1, wherein the autophagy modulating agent is an autophagy enhancing agent or an autophagic pathway modulating agent.

10. The composition of claim 1, wherein the autophagy modulating agent is used to treat an autophagy-associated disease, disorder, or condition.

11. A method of modulating autophagy or treating or preventing an autophagy-associated disease, disorder, or condition in a subject in need thereof, comprising administering to the subject a pharmaceutical composition comprising an autophagy modulating agent selected from formula (I): 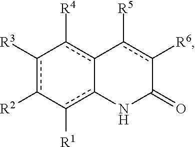
wherein R¹, R², R³, R⁴, R⁵, or R⁶ is hydrogen (H), halo (e.g., Cl, F), C₁₋₈alkyl (e.g., methyl, ethyl, butyl, propyl, isopropyl, isopentyl), halogen-substituted C₁₋₈alkyl (e.g., trifluoromethyl), piperidinyl (e.g., piperidin-1, 2, 3, or 4-yl), C₃₋₁₀cycloalkyl (e.g., phenyl), halogen-substituted C₃₋₁₀cycloalkyl (e.g., chlorophenyl), pyrimidinyl (e.g., pyrimidin-2,4, or 5, -yl), C₁₋₈alkoxy (e.g., methoxy, ethoxy, propoxy, butoxy, hexoxy), halogen substituted C₁₋₈alkyl (e.g., trifluoromethyl, trifluorobutoxy, trifluoropentoxy), C₁₋₆alkyl-C₃₋₁₀cycloalkoxy (e.g., cyclopropylethoxy), C₃₋₁₀cycloalkyl-C₁₋₆alkyl-amino (e.g., cyclopropylethylamino, cyclopropylethyl(methyl)amino), C₃₋₁₀heterocyclyloxy (e.g., piperidinyloxy, cyclopentylpiperidinyloxy), C₁₋₈alkylsulphonyl (e.g., methyl sulfonyl), C₁₋₈alkylsulphonylalkoxy (e.g., methyl sulfonylpropoxy), C₁₋₁₀cycloalkoxy (e.g., phenyl propoxy), alkoxyC₁₋₁₀cycloalkyl (e.g., methoxy phenyl), substituted phenyl; and/or
R¹ or R², R² or R³, or R³ or R⁴ optionally form a ring (e.g., heterocyclyl, cycloalkyl, furanyl, pyridinyl, phenyl, etc.); a 5 or 6 membered ring (e.g., pyridinyl, furanyl, cycloalkyl, heterocyclyl); a substituted 5- or 6-membered ring (e.g., an alkyl-, alkyl-phenyl-, hydroxyl-, halo-, chloro-, bromo-, methoxy-, di-methoxy-, oxy-, or benzyl-substituted 5 or 6 membered ring); or a substituted bicyclic group (e.g., indole, azobicyclo, bridged bicyclic).

12. The method of claim 11, wherein the autophagy modulating agent is selected from:
(i) a 5,6-disubstituted quinolone;

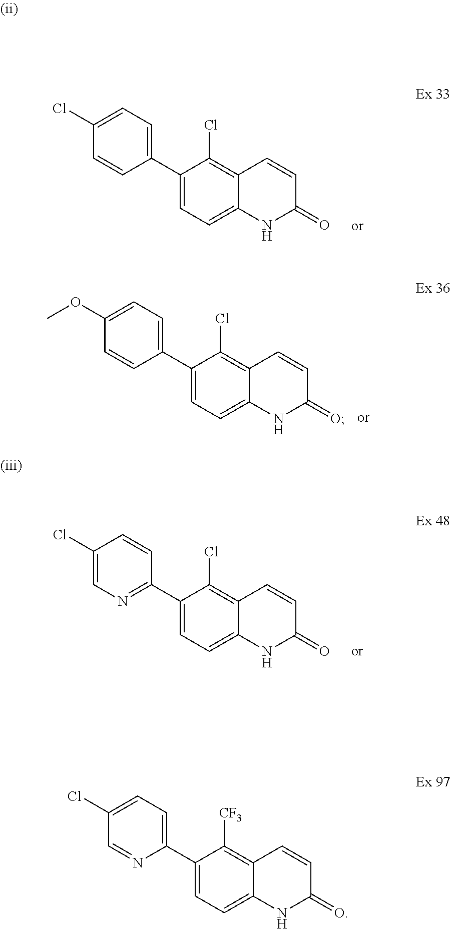 [SMILES: O=c1ccc2c(Cl)c(-c3ccc(Cl)cc3)ccc2[nH]1 ; COc1ccc(-c2ccc3[nH]c(=O)ccc3c2Cl)cc1 ; "or" ; O=c1ccc2c(Cl)c(-c3ccc(Cl)cn3)ccc2[nH]1 ; O=c1ccc2c(C(F)(F)F)c(-c3ccc(Cl)cn3)ccc2[nH]1]

13. The method of claim 12, wherein the autophagy modulating agent is a 5,6-disubstituted quinolone.

14. The method of claim 13, wherein the autophagy modulating agent is

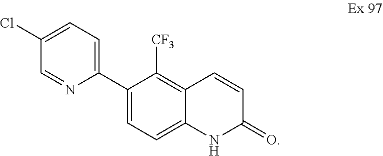 [SMILES: O=c1ccc2c(C(F)(F)F)c(-c3ccc(Cl)cn3)ccc2[nH]1]

15. The method of claim 11, wherein the autophagy modulating agent is selected from the group consisting of: 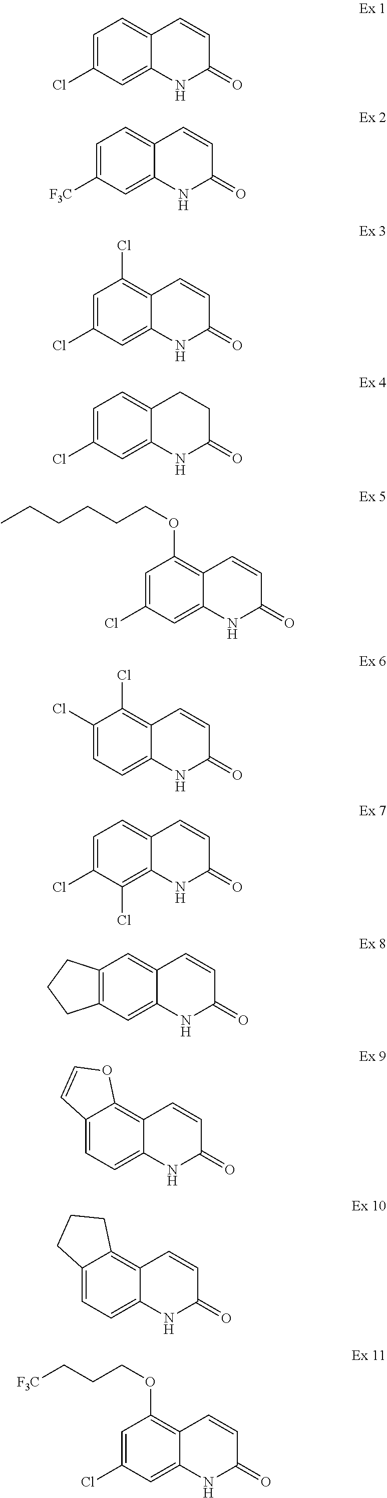 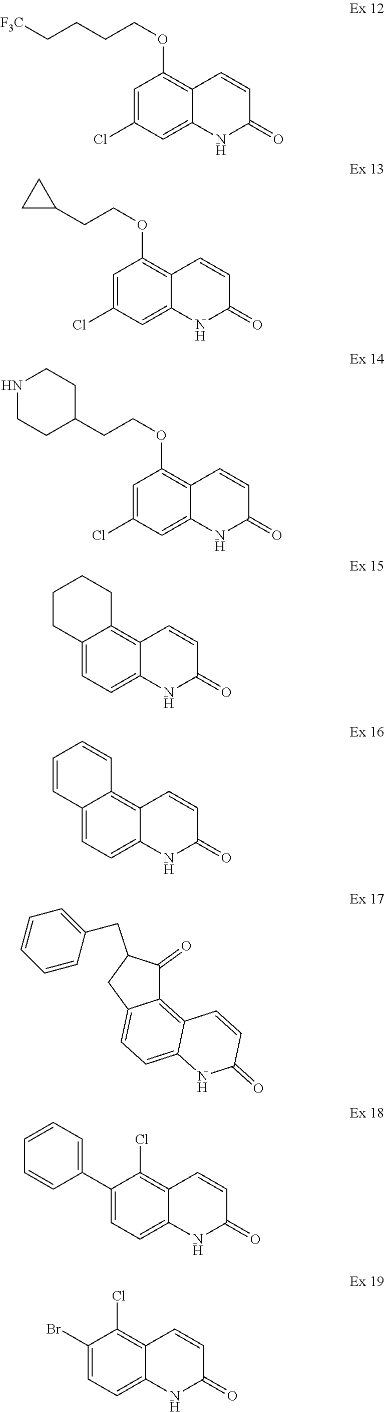 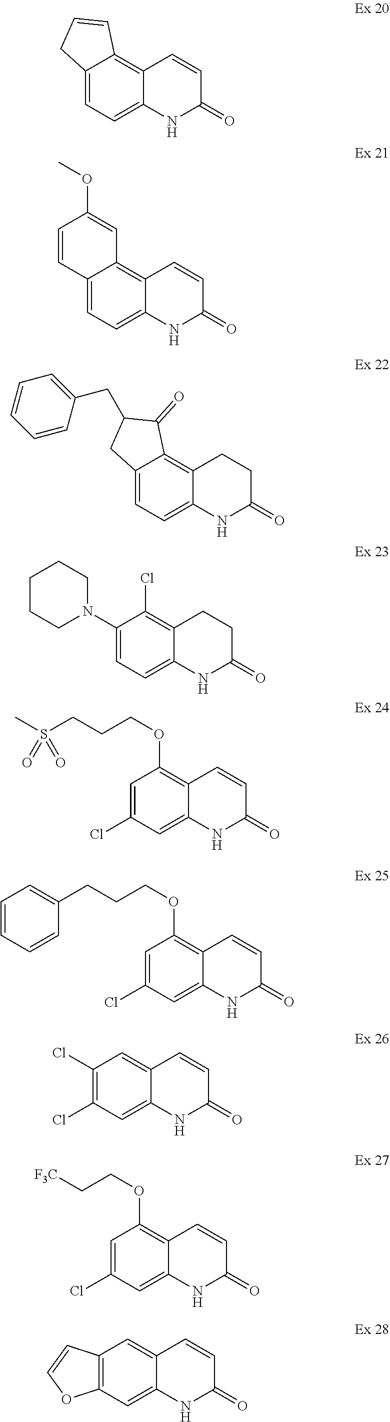 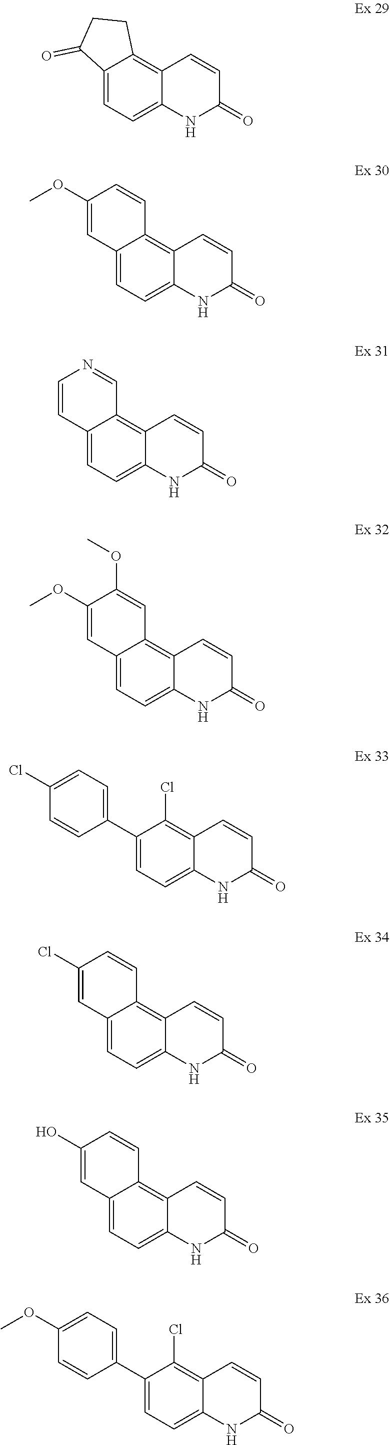 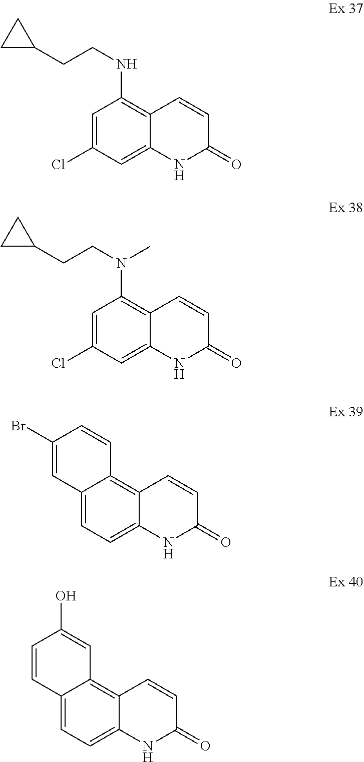 [SMILES: O=c1ccc2c(NCCC3CC3)cc(Cl)cc2[nH]1 ; CN(CCC1CC1)c1cc(Cl)cc2[nH]c(=O)ccc12 ; O=c1ccc2c(ccc3cc(Br)ccc32)[nH]1 ; O=c1ccc2c(ccc3ccc(O)cc32)[nH]1]
or a pharmaceutically acceptable salt thereof, including all tautomers and stereoisomers, and optionally substituted analogs thereof.

16. The method of claim 11, wherein the autophagy modulating agent is selected from the group consisting of: 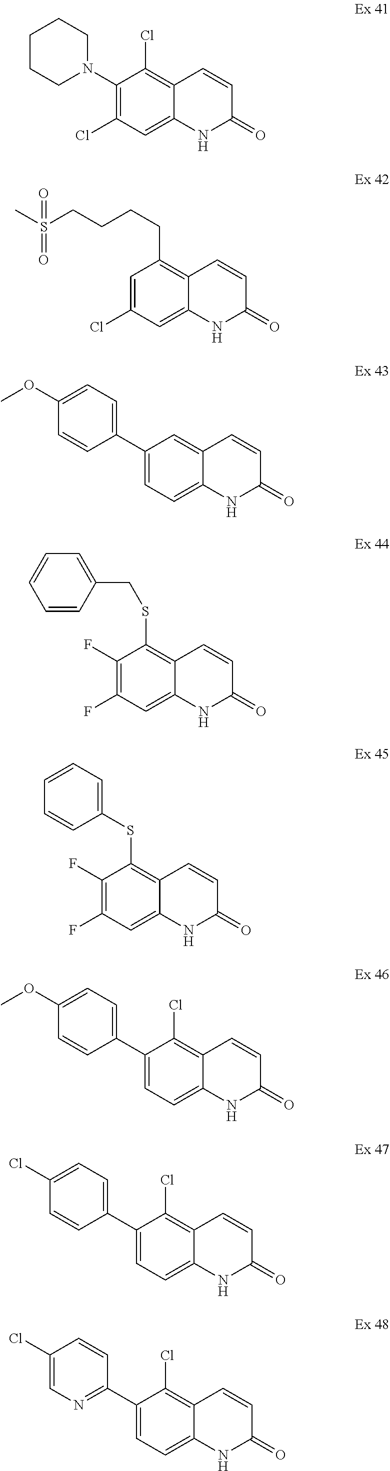 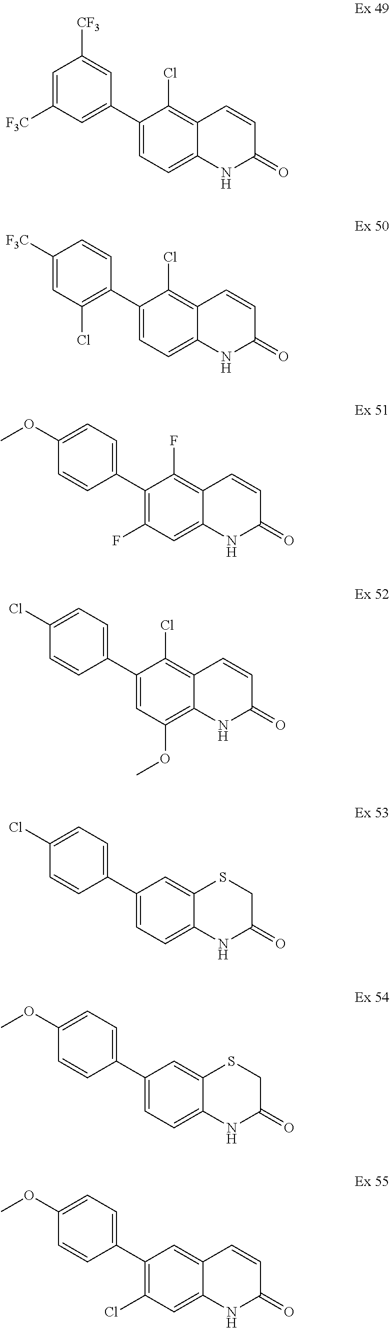 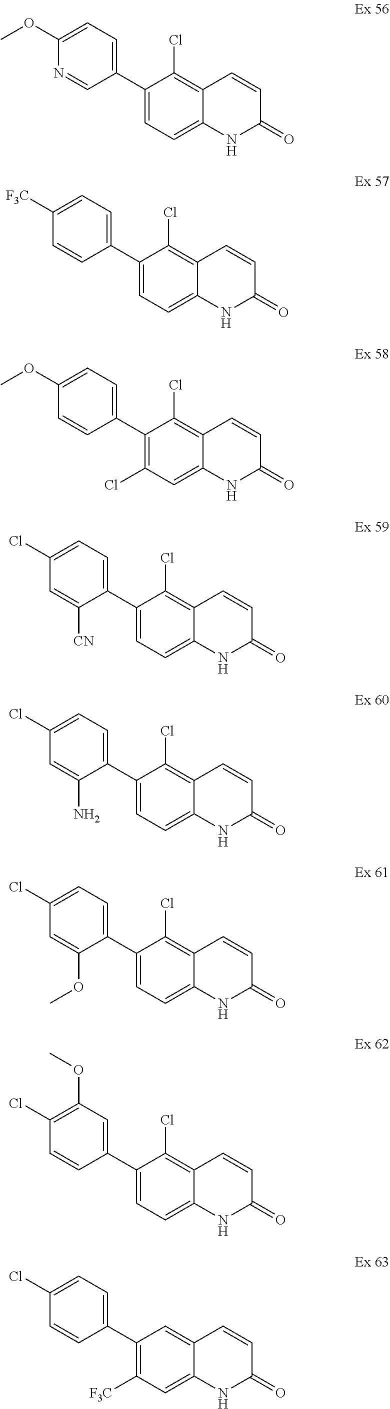 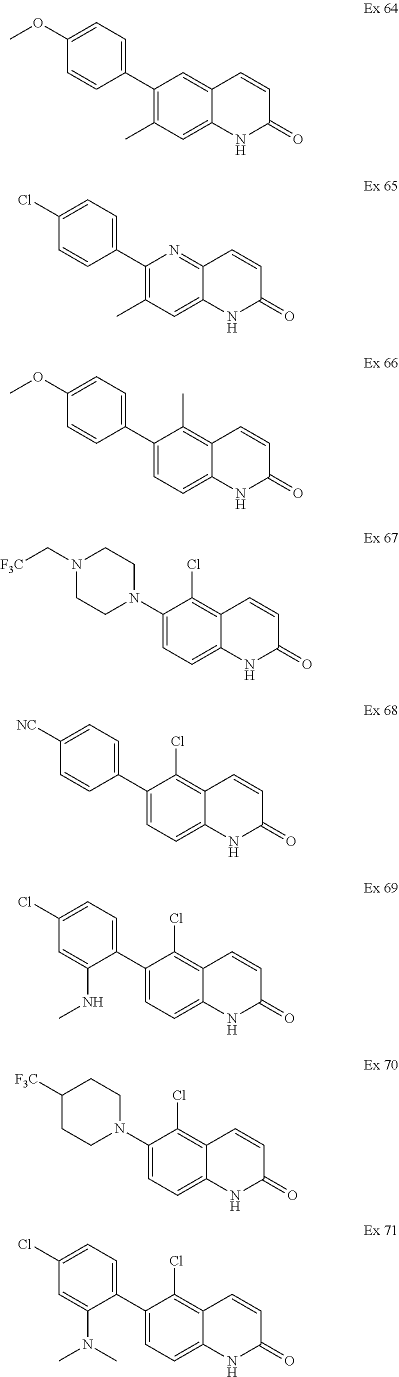 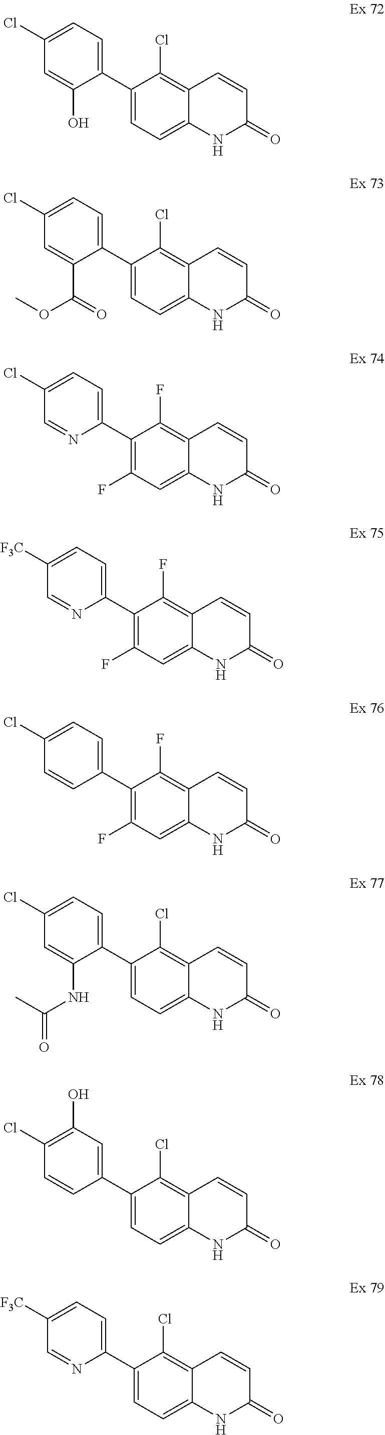 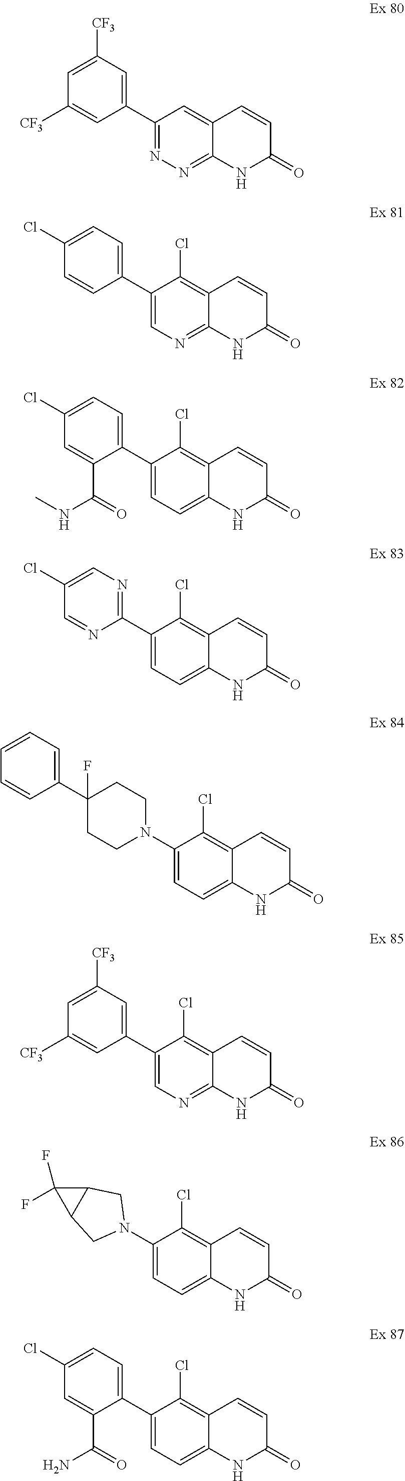 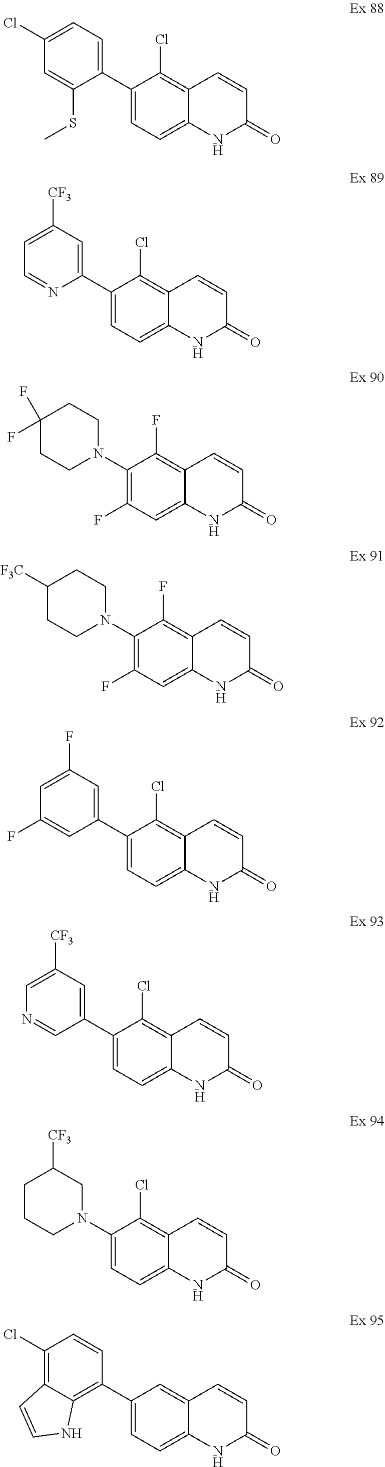 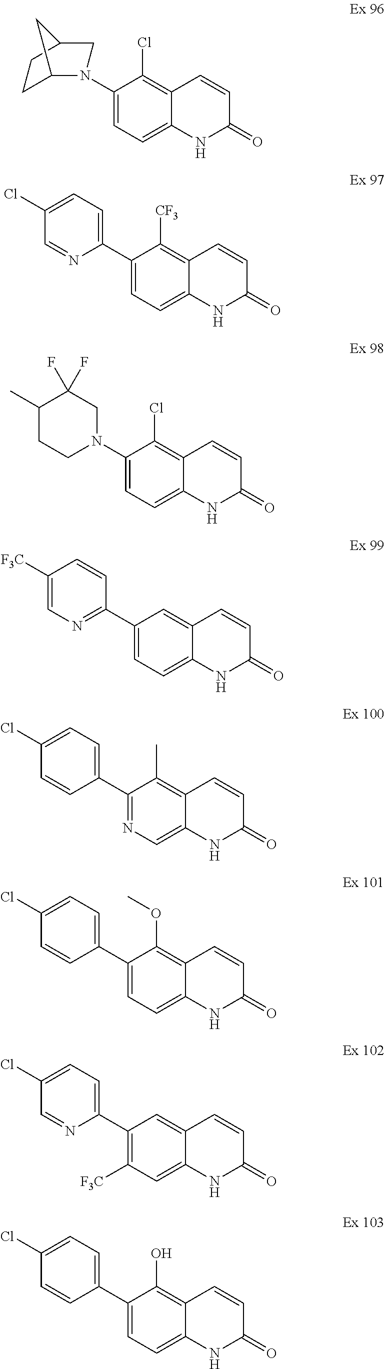 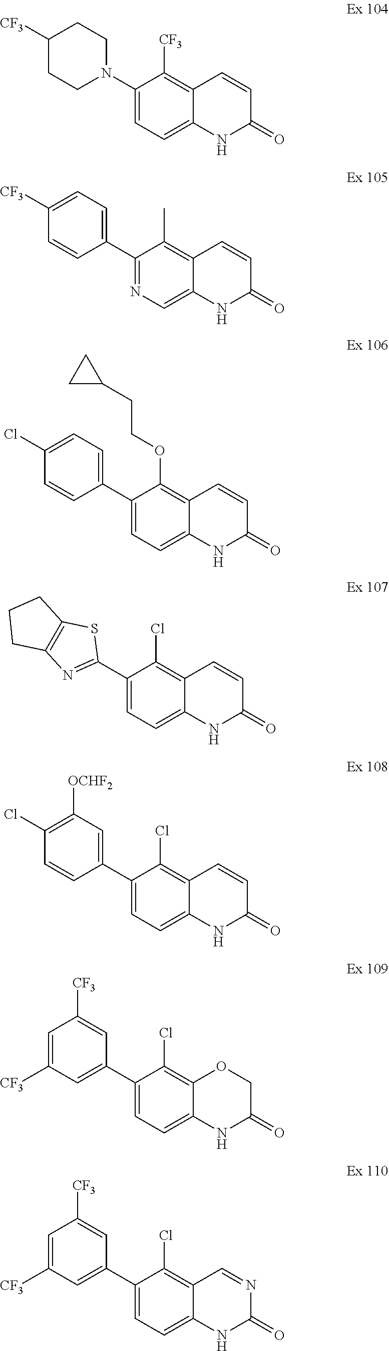 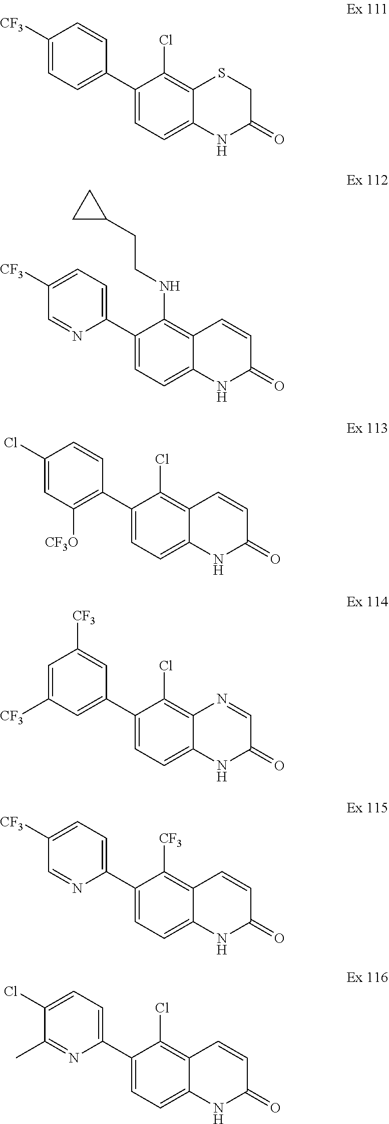
or a pharmaceutically acceptable salt thereof, including all tautomers and stereoisomers, and optionally substituted analogs thereof.

17. The method of claim 11, wherein R¹ is hydrogen.

18. The method of claim 11, wherein the autophagy modulating agent has autophagy modulating activity.

19. The method of claim 11, wherein the autophagy modulating agent is an autophagy enhancing agent or an autophagic pathway modulating agent.

20. The method of claim 11, wherein the subject has or is suspected of having an autophagy-associated disease, disorder, or condition.

21. The method of claim 20, wherein the autophagy modulating agent inhibits progression of the autophagy-associated disease, disorder, or condition.

22. The method of claim 20, wherein the autophagy-associated disease, disorder, or condition is selected from alpha-1 antitrypsin deficiency (ATD), liver disease from ATD, an age-dependent degenerative disease, Alzheimer's disease (AD), inherited emphysema, diabetes, cancer, or a polyglutamine (polyQ) disease.

23. The method of claim 22, wherein the polyglutamine (polyQ) disease is selected from Huntington's disease (HD); spinocerebellar ataxias (SCA) types 1, 2, 6, 7, or 17; Machado-Joseph disease (MJD/SCA3); dentatorubral pallidoluysian atrophy (DRPLA); spinal and bulbar muscular atrophy; or X-linked 1 (SMAX1/SBMA).

24. The method of claim 11, wherein the autophagy modulating agent reduces cellular accumulation of misfolded or aggregated proteins.

25. The method of claim 24, wherein the autophagy modulating agent reduces aggregated alpha-1 antitrypsin Z variant (ATZ) protein.

26. The method of claim 24, wherein the autophagy modulating agent reduces or prevents accumulation of misfolded protein in liver cells, liver damage, liver fibrosis, or liver failure.

27. The method of claim 11, wherein the autophagy modulating agent has anti-tumor activity in a subject having cancer.

28. The method of claim 11, wherein the autophagy modulating agent reduces neuronal cell death.

29. The method of claim 11, wherein the autophagy modulating agent treats or prevents hepatic fibrosis.

30. The method of claim 11, wherein the autophagy modulating agent reduces liver fibrosis.

31. The method of claim 11, wherein the autophagy modulating agent does not significantly affect insulin secretion.
